(19) United States
(12) Patent Application Publication
(10) Pub. No.: US 2022/0251640 A1
(43) Pub. Date: Aug. 11, 2022

(54) METHODS OF DETERMINATION OF GENOME ARCHITECTURE AND EPIGENETIC PROFILE

(71) Applicants: THE BROAD INSTITUTE, INC., Cambridge, MA (US); BAYLOR COLLEGE OF MEDICINE, Houston, TX (US)

(72) Inventors: Erez AIDEN, Houston, TX (US); Elena STAMENOVA, Cambridge, MA (US); Andreas GNIRKE, Cambridge, MA (US); Eric S. LANDER, Cambridge, MA (US); Neva DURAND, Houston, TX (US); Olga DUDCHENKO, Houston, TX (US)

(21) Appl. No.: 17/612,160

(22) PCT Filed: May 18, 2020
(86) PCT No.: PCT/US2020/033436

§ 371 Date: Nov. 17, 2021

Related U.S. Application Data
(60) Provisional application No. 62/849,615, filed on May 17, 2019.

(51) Int. Cl. C12Q 1/6869; C12Q 1/6806

(52) U.S. Cl. CPC C12Q 1/6869; C12Q 2600/154; C12Q 1/6806

(57) ABSTRACT

Methods for analyzing spatial proximity and epigenetic profile of nucleic acids in cells, the method comprising: fragmenting the nucleic acids, the fragmented nucleic acids comprising overhanging ends; filling in the overhanding ends with one or more labeled nucleotides; joining the filled in ends to create one or more end-joined nucleic acid fragments with one or more junctions; treating the end-joined nucleic acid fragments with bisulfite; isolating the bisulfite-treated end-joined nucleic acid fragments using the label; and determining sequence at the one or more junctions in the bisulfite treated end-joined nucleic acid fragments, thereby determining spatial proximity between the nucleic acids and the methylation profile of the nucleic acids.

### CROSS-REFERENCE TO RELATED APPLICATIONS

This application claims the benefit of U.S. Provisional Application No. 62/849,615, filed May 17, 2019. The entire contents of the above-identified application are hereby fully incorporated herein by reference.

### STATEMENT REGARDING FEDERALLY SPONSORED RESEARCH

This invention was made with government support under Grant Nos. OD008540, HG009375, and OD008540 awarded by the National Institutes of Health, and Grant No. 2017-05741 awarded by National Institute of Food and Agriculture. The government has certain rights in the invention.

### TECHNICAL FIELD

The subject matter disclosed herein is generally directed to determining genome architecture and epigenetic profile.

### BACKGROUND

Nuclear architecture and DNA methylation play critical and interdependent roles in mammalian genome regulation. For example, the insulator protein CTCF, which interacts with the cohesin complex to form chromatin loops and thereby establish discrete structural and functional segments of the genome, binds in a methylation-sensitive fashion. Similarly, a recent study has implicated hyper-methylation induced disruption of chromosome topology in oncogene activation5.

Nuclear architecture and DNA methylation may be interrogated independently using Hi-C proximity ligation mapping and whole-genome bisulfite sequencing (WGBS), respectively. However, independent assays of architecture and methylation can obscure the interdependence of the underlying phenomena. In addition, since both sequencing assays require deep coverage of the genome, performing them separately increases the cost. Thus, there is a need for methods for simultaneously determining nuclear architecture and DNA methylation.

### SUMMARY

In one aspect, the present disclosure provides methods for analyzing nucleic acids in cells, comprising fragmenting the nucleic acids, the fragmented nucleic acids comprising overhanging ends; filling in the overhanging ends with one or more nucleotides comprising a label; joining the filled in ends to create one or more end joined nucleic acid fragments with one or more junctions; treating the end-joined nucleic acid fragments with bisulfite; isolating the bisulfite-treated end-joined nucleic acid fragments using the label; and determining sequence at the one or more junctions in the bisulfite treated end-joined nucleic acid fragments, thereby determining spatial proximity between the nucleic acids and the methylation profile of the nucleic acids.

In some embodiments, the method further comprises determining a relationship between the spatial proximity and the methylation profile. In some embodiments, the method further comprises holding the nucleic acids in a fixed position relative to one another prior to fragmenting. In some embodiments, the nucleic acids are held in the fixed position by crosslinking the cells or nuclei in the cells. In some embodiments, the method further comprises reversing the crosslinking. In some embodiments, the method further comprises isolating nuclei from the cells prior to fragmenting. In some embodiments, the method further comprises permeabilizing the nuclei. In some embodiments, the nucleic acids are a part of chromatin. In some embodiments, the nucleic acids are DNA. In some embodiments, fragmenting is performed by digesting the nucleic acids using a nuclease. In some embodiments, the nuclease is methylation insensitive. In some embodiments, the method further comprises, prior to the bisulfite treatment, shearing the nucleic acids.

In some embodiments, the sheared nucleic acids have a length from about 300 base pairs (bp) to about 500 bp. In some embodiments, the bisulfite treated end-joined nucleic acid fragments are isolated using a capture agent that binds to the labeled nucleotides. In some embodiments, the capture agent is attached to a solid support. In some embodiments, the solid support is a bead. In some embodiments, the method further comprises attaching one or more adaptors to the bisulfite treated, end-joined nucleic acid fragments. In some embodiments, the one or more adaptors are attached after isolating the bisulfite treated, end-joined nucleic acid fragments. In some embodiments, the method further comprises amplifying the bisulfite treated, end-joined nucleic acid fragments. In some embodiments, the bisulfite treated, end-joined nucleic acid fragments are amplified using primers with one or more barcodes. In some embodiments, the method further comprises quantifying a frequency with which pairs of loci in the nucleic acids are found adjacent, and a frequency with which loci in the nucleic acids are methylated.

In some embodiments, determining the spatial proximity between the nucleic acids comprises identifying chromosomal location of nucleic acid sequences both 5′ and 3′ of the junctions. In some embodiments, determining the methylation profile comprises generating a genome-wide methylation profile of the cells. In some embodiments, the method further comprises correlating a relationship between the spatial proximity and the methylation profile with a disease. In some embodiments, the sequence at one or more junctions in the bisulfite treated, end-joined nucleic acid fragments is determined by transporting the fragments through an orifice in an electric field and measuring change of an electric current density across the orifice when the fragments are transported. In some embodiments, the sequence at the one or more junctions in the bisulfite treated, end-joined nucleic acid fragments is determined by nanopore sequencing.

In another aspect, the present disclosure provides a method of diagnosing a disease in a subject, comprising obtaining cells from the subject; analyzing nucleic acids in the cells according to the method herein, wherein the spatial proximity and the methylation profile are indicative of the disease in the subject.

In another aspect, the present disclosure provides a method of treating a disease in a subject, comprising: determining spatial proximity and methylation profile of a gene in a cell from the subject; comparing the spatial proximity and the methylation profile to reference values, thereby identifying one or more nucleotides in the gene related to the disease; and modifying at least one of the identified nucleotides.

In some embodiments, the spatial proximity and the methylation profile of the one or more identified nucleotides are indicative of the disease. In some embodiments, modifying at least one of the identified nucleotides comprises modifying methylation of the at least one of the identified nucleotides. In some embodiments, modifying at least one of the identified nucleotides comprises converting at least one of the identified nucleotides to another nucleotide.

In another aspect, the present disclosure provides a method for screening chemical libraries for agent modulating chromatin architecture and epigenetic profiles, comprising exposing cells to members of the chemical libraries; determining the spatial proximity and methylation profile of according to methods herein; and comparing the spatial proximity and the methylation profile to spatial proximity and methylation profile of control cells, thereby identifying members in the chemical libraries that have effects on the spatial proximity and methylation profile.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1E—Mapping chromatin contacts and DNA methylation simultaneously by Hi-Culfite. (FIG. 1A) Left: the initial steps were identical to standard in situ Hi-C. Right: the “bisulfite first” protocol entailed capture of biotin-marked bisulfite-converted ligation products and the use of a commercial kit for preparing sequencing libraries from bisulfite-converted DNA. (FIG. 1B) Overview of the JuiceMe pipeline for processing of Hi-Culfite sequencing data. The software was built off of Juicer (20); reads were aligned as single end via bwa-meth (22), chimeric reads were handled by either pairing appropriately or removal, reads were sorted and merged, and duplicates and near duplicates were removed. Simultaneously, all reads that map were merged into a bam, duplicates marked, and the software MethylDackyl (github.com/dpryan79/MethylDackel) was used to create the CpG methylation track. (FIG. 1C, FIG. 1D) Comparison of GM12878 (FIG. 1C) and IMR90 (FIG. 1D) chromatin contact maps and DNA methylation tracks of human chromosome 14q obtained in a single Hi-Culfite experiment (left) or by separate in situ Hi-C and WGBS assays (right). Chromosome-scale contact matrices had 1 Mb resolution. Zoom-ins had 50 kb and 5 kb resolution. CpG methylation tracks are shown above each Hi-C contact matrix. Eigenvectors denoting open (positive values) and closed chromatin compartments (negative) along the chromosome are shown at the top. (FIG. 1E) Smoothed scatter plots (bottom left), Pearson correlation coefficients for pair-wise comparisons (top right) and bimodal distributions of single-CpG DNA methylation values (diagonal). ENCODE rep1 and rep2 were biological WGBS replicates. The Hi-Culfite data were from combined 2 technical replicates prepared from the same batch of cross-linked GM12878 cells (Hi-Culfite Replicate 1) and one biological replicate derived from different expansion of the same cell line (Hi-Culfite Replicate 2). The scatter plot was produced by MethylKit (23); the figure shows the linear regression fit and the polynomial regression fit.

FIGS. 2A-2B. The methylation frequency of a locus varied as a function of its chromatin neighborhood in a particular cell. (FIG. 2A) The methylation frequency of a locus varied as a function of its chromatin neighborhood. The figure shows deviation between the genome-wide methylation frequency for a 500-kb locus, and its observed methylation frequency when in contact with a particular neighbor locus. Elevated values are shown in light grey, and diminished values in dark grey. Loci in the A compartment were more likely to be methylated when in contact with a locus in the B compartment. (FIG. 2B) Methylation frequency as a function of the genomic position of the neighbor locus and its methylation state. When the neighbor locus was methylated, the methylation frequency at the index locus is indicated using the light grey bars; when the neighbor was unmethylated, the methylation frequency at the index locus is indicated using the dark grey bars. The dashed line indicates the average methylation frequency at that locus.

FIGS. 3A-3C—Comparison of Hi-Culfite data to separate WGBS and Hi-C data sets. (FIG. 3A) CpG read coverage percentage in GM12878 WGBS data from two replicates generated by ENCODE Consortium and two replicates of Hi-Culfite of GM12878. (FIG. 3B) Low sequencing depth comparison of contact maps, DNA methylation tracks and eigenvectors indicating open (positive values) and closed (negative values) chromatin compartments along chromosome 14 of Hap1 cell line generated by single Hi-Culfite experiment (left) and separated in situ Hi-C and WGBS assays (right). (FIG. 3C) Scatter plot style heat map (bottom left), Pearson correlation coefficients for pair-wise comparisons (top right) and bimodal distributions of single-CpG DNA methylation values (diagonal) for Hap1 and IMR90. Hi-Culfite data generated for both cell lines was compared with respect to WGBS was from HapI and ENCODE WGBS data for IMR90. This plot was produced by MethylKit (7).

FIGS. 4A-4B. Hi-Culfite data shows that DNA methylation inhibition by 5-azacytidine has no effect on chromosome-level nuclear organization in GM12878 and Hap1 cell lines. (FIG. 4A) Hi-Culfite contact maps of chromosome 14 and eigenvectors indicating open (positive values) and closed (negative values) chromatin compartments of GM12878 and Hap1 cells treated for 8 days with DMSO (control) or increasing concentration (1 uM and 5 uM) of 5-azacytidine show no difference despite of global decrease in DNA methylation (methylation tracks in dark grey) (FIG. 4B) Aggregate Peak Analysis performed on the Hi-C maps using loop lists for GM12878 and HapI showed no significant changes in looping signal with increasing 5-azacytidine concentration. In order to assess the potential formation of new convergent CTCF loops with 5-azacytidine treatment, loop lists from the other cell type were also used for APA analysis on the alternative map.

FIGS. 5A-5B—RAD21 Degron Hi-Culfite experiments recapitulated loss of CTCF-mediated loops. (FIG. 5A) Hi-Culfite contact maps of chromosome 14 and eigenvectors indicating open (positive values) and closed (negative values) chromatin compartments of HCT-116 before and after auxin treatment. (FIG. 5B) Aggregate Peak Analysis performed at 10 kB resolution using the HCT-116 loop list showed loss of loops with auxin treatment.

FIGS. 6A-6B—Comethylation analysis shows that Hi-C contacts share methylation status more frequently than expected from the null hypothesis. (FIG. 6A) Difference of observed comethylation matrix and expected comethylation matrix on chromosome q14 at 1 Mb. The difference indicates that Hi-C contacts tend to share the same methylation status. (FIG. 6B) Difference of the observed methylation correlation matrix and the observed unmethylation correlation matrix. A methylated locus is more likely to depend on its spatial context than an unmethylated locus.

FIGS. 7A-7B—Aggregate methylation analysis recapitulated CTCF motif site relationships with nucleosome occupancy and methylation asymmetries. (FIG. 7A) Aggregation of methylation data from the combined Hi-Culfite GM12878 replicates 1 and 2 at oriented CTCF motifs recapitulated nucleosome occupancy and directional methylation asymmetry. (FIG. 7B) Aggregation of methylation data from 5-azacytidine experiments shows no changes to nucleosome occupancy and directional methylation asymmetry with increasing concentration of 5-azacytidine.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides methods for determining genome architecture and epigenetic profiles. The methods herein allow simultaneous determination of spatial proximity relationship between nucleic acids and epigenetic profile of the nucleic acids. The correlation of the spatial proximity and the epigenetic profile (e.g., methylation profile) may be assessed and used for diagnosing and treating diseases.

In general, the methods herein comprise fragmenting nucleic acids to create overhanging ends, filling in the overhangs with labeled nucleotides, joining the filled ends to create junctions, treating the enjoined nucleic acids with bisulfite, isolating and analyzing the bisulfite-treated nucleic acids. The results from the analysis may be used for characterizing chromatin architecture and epigenetic profile of genes of interests. Such information may be used for diagnosing diseases and/or planning treatment for the diseases. In some examples, the methods comprise one or more of fragmenting the nucleic acids, the fragmented nucleic acids comprising overhanging ends; filling in the overhanging ends with one or more labeled nucleotides; joining the filled in ends to create one or more end-joined nucleic acid fragments with one or more junctions; treating the end-joined nucleic acid fragments with bisulfite; isolating the bisulfite-treated, end-joined nucleic acid fragments using the label; and determining sequence at the one or more junctions in the bisulfite treated end-joined nucleic acid fragments, thereby determining spatial proximity relationships between the nucleic acids and the methylation profile of the nucleic acids.

### Methods of Analysis

The methods herein may be used for analyzing one or more features of nucleic acids. The methods disclosed comprise the steps of fragmenting the nucleic acids thereby generating fragmented nucleic acids comprising overhanging ends; filling in the overhanging ends of the fragmented nucleic acids with one or more nucleotides comprising a label; joining the filled-in ends of the fragmented nucleic acids to create one or more end-joined nucleic acid fragments with one or more junctions; treating the end joined nucleic acid fragments with bisulfite; isolating the bisulfite-treated end-joined nucleic acid fragments using the label; and determining the sequence at the one or more junctions in the bisulfite treated end-joined nucleic acid fragments, thereby determining spatial proximity between the nucleic acids and the methylation profile of the nucleic acids.

### Fragmenting Nucleic Acids

The methods herein may comprise fragmenting nucleic acids. In some embodiments, in order to create discrete portions of nucleic acid that can be joined together in subsequent steps of the methods, the nucleic acids present in the cells, such as cross-linked cells, are fragmented. In one example embodiment, the fragmentation may be done enzymatically. In another example embodiment, the fragmentation may be done chemically.

### Overhanging Ends

For example, DNA can be fragmented using an enzyme (e.g., an endonuclease) that cuts a specific sequence of DNA and leaves behind a DNA fragment with an overhang, thereby yielding fragmented DNA.

When a nuclease cleaves DNA asymmetrically, a stretch of single stranded nucleotides is left. In some cases, the overhang is a 5′ overhang. In certain cases, the overhang is a 3′ overhang. In other examples an endonuclease can be selected that cuts the DNA at random spots and yields overhangs or blunt ends. In some embodiments, fragmenting the nucleic acid present in the one or more cells comprises enzymatic digestion with an endonuclease that leaves 5′ overhanging ends. Enzymes that fragment, or cut, nucleic acids and yield an overhanging sequence are known in the art and can be obtained from such commercial sources as New England BioLabs® and Promega®. One of ordinary skill in the art will appreciate that using different fragmentation techniques, such as different enzymes with different sequence requirements, will yield different fragmentation patterns and therefore different nucleic acid ends. The process of fragmenting the sample can yield ends that are capable of being joined.

In some examples, the endonuclease for nucleic acid fragmentation is a methylation-sensitive endonuclease. A “methylation-sensitive endonuclease” refers to a restriction enzyme that cleaves at or in proximity to an unmethylated recognition sequence but does not cleave at or in proximity to the same sequence when the recognition sequence is methylated. Exemplary 5′-methyl cytosine sensitive endonuclease include, e.g., Aat II, Aci I, Ad L Age L Alu L Asc L Ase I, AsiS I, Bbe I, BsaA I, BsaH I, BsiE I, BsiW I, BsrF I, BssH II, BssK I, BstB I, BstN I, BstU I, Cla I, Eae I, Eag I, Fau I, Fse I, Hha I, HinP1 I, HinC II, Hpa II, Hpy99 I, HpyCH4 IV, Kas I, Mlu I, MapAl I, MboI, Msp I, Nae I, Nar I, Not I, Pml I, Pst I, Pvu I, Rsr II, Sac II, Sap I, Sau3A I, Sfl I, Sfo I, SgrA I, Sma I, SnaB I, Tsc I, Xma I, or Zra I. In one example, the endonuclease used herein is MboI.

In some examples, the endonuclease for nucleic acid fragmentation is a methylation-dependent endonuclease. A “methylation-dependent endonuclease” refers to a restriction enzyme that cleaves at or near a methylated recognition sequence, but does not cleave at or near the same sequence when the recognition sequence is not methylated. Methylation-dependent endonuclease can recognize, for example, specific sequences comprising a methylated-cytosine or a methylated-adenosine. Methylation-dependent restriction enzymes include those that cut at a methylated recognition sequence (e.g., DpnI) and enzymes that cut at a sequence that is not at the recognition sequence (e.g., McrBC). Exemplary methylation-dependent endonucleases include, e.g., McrBC, McrA, MrrA, and Dpn I. One of skill in the art will appreciate that homologs and orthologs of the restriction enzymes described herein are also suitable for use in the present invention.

In some examples, the endonuclease for nucleic acid fragmentation is a methylation insensitive endonuclease. A “methylation insensitive endonuclease” refers to a restriction enzyme that cuts DNA regardless of the methylation state of the base of interest (A or C) at or near the recognition sequence. In some examples, the endonuclease for nucleic acid fragmentation is a methylation sensing endonuclease. A “methylation sensing endonuclease” refers to a restriction enzyme whose activity changes in response to the methylation of its recognition sequence

### Nucleic Acids

The methods herein may be used for analyzing one or more features of nucleic acids. The nucleic acids may be deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand.

In some examples, the nucleic acids are genomic DNA. In certain examples, the nucleic acids are components of chromatins. Chromatin may be a complex of molecules including proteins and polynucleotides (e.g. DNA, RNA), as found in a nucleus of a eukaryotic cell. Chromatin may comprise histone proteins that form nucleosomes, genomic DNA, and other DNA binding proteins (e.g., transcription factors) that are generally bound to the genomic DNA. The chromatins may be in nuclei (e.g., isolated nuclei). In some cases, the chromatin may be isolated chromatin, e.g., from lysed nuclei.

In some embodiments, the methods may be used for analyzing a sample of DNA where all copies of a genomic DNA locus have an identical methylation pattern. In other embodiments, the DNA sample is a mixture of DNA comprising alleles of a DNA locus in which some alleles are more methylated than others. In some embodiments, a DNA sample contains DNA from two or more different cell types, wherein each cell type has a different methylation density at a particular locus. For example, at some loci, neoplastic cells have different methylation densities compared to normal cells. If a tissue, body fluid, or secretion contains DNA from both normal and neoplastic cells, then the DNA sample from the tissue, body fluid, or secretion will comprise a heterogeneous mixture of differentially methylated alleles. In this case, at a given locus, one set of alleles within the DNA (e.g., those derived from neoplastic cells in the sample) will have a different methylation density than the other set of alleles (e.g., those derived from normal cells).

In mixed samples (e.g., in biopsies comprising healthy and diseased cell), it may be helpful to focus results on one population of nucleic acids in the sample (e.g., from diseased cells) rather than to determine the average methylation density across DNA from all cells in the sample. In some embodiments in which a first population of DNA in the sample has low or no methylation and the second population of DNA in the sample has more methylation than the first population, density in the second population can be determined by cleaving the sample with one or more methylation-sensitive restriction enzymes (generally cut to “completion”), thereby degrading the first population while leaving the second population substantially intact. Thus, the sample may also be contacted with a methylation-dependent restriction enzyme (using McrBC and/or any methylation-dependent restriction enzyme under partial digestion conditions) and the remaining intact DNA may be amplified, thereby determining the methylation density in the second population. The methylation density of the first population may be similarly determined by contacting the sample with one or more methylation-dependent restriction enzymes (generally cut to “completion”) and contacting the sample with a methylation sensitive under partial digestion conditions. In this case, the amplified DNA will represent the methylation density of the first population.

### Filling in Overhangs

The methods may further comprise filling in the overhangs in the fragmented nucleic acids. The overhangs may be filled in with nucleotides using a polymerase (e.g., a DNA polymerase). In some cases, the filled in nucleic acid fragments are blunt ended at the filled end (e.g., 5′ end).

### Labeled Nucleotides

In some cases, the overhangs are filled in with one or more labeled nucleotides, e.g., nucleotides comprising label(s). The labeled nucleotides may be used to identify and/or isolate the filled in ends in later step. In cases where the filled in ends are joined, the labeled nucleotides may be used to identify and/or isolate the joined ends.

Labels in the nucleotides may be used for isolating the nucleic acid to which the labeled nucleotides incorporate. Examples of such labels include biotin, aminoallyl-labeled nucleotides, sulfhydryl-labeled nucleotides, allyl- or azide-containing nucleotides, and many other methods described in Bioconjugate Techniques (2nd Ed), Greg T. Hermanson, Elsevier (2008), which is specifically incorporated herein by reference.

### End Joining

The methods herein may further comprise joining the ends of the fragmented nucleic acids. In some embodiments, the fragmented nucleic acids are end joined at the filled in ends, for example, by ligation using a nucleic acid ligase (e.g., T4 ligase), or otherwise attached to another fragment that is in close physical proximity. The ligation, or other attachment procedure, for example nick translation or strand displacement, creates one or more end joined nucleic acid fragments having a junction, for example a ligation junction, wherein the site of the junction, or at least within a few bases, includes one or more labeled nucleic acids, for example, one or more fragmented nucleic acids that have had their overhanging ends filled and joined together. While this step typically involves a ligase, it is contemplated that any means of joining the fragments can be used, for example, any chemical or enzymatic means. Further, it is not necessary that the ends be joined in a 3′-5′ ligation.

The joined ends may create a junction, which is a site where two nucleic acid fragments are joined, for example using the methods described herein. A junction may contain information about the proximity of the nucleic acid fragments that participate in formation of the junction. For example, junction formation between two nucleic acid fragments indicates that these two nucleic acid sequences were in close proximity when the junction was formed, although they may not be in proximity in liner nucleic acid sequence space. Thus, a junction can define line range interactions. In some embodiments, a junction is labeled, for example with a labeled nucleotide, for example to facilitate isolation of the nucleic acid molecule that includes the junction.

The end joined nucleic acid fragments may be between about 100 and about 1000 bases in length, although longer and shorter fragments are also contemplated. In some embodiments, the nucleic acid fragments are from about 100 to about 1000 bases in length, such as about 100, about 150, about 200, about 250, about 300, about 350, about 400, about 450, about 500, about 550, about 600, about 650, about 700, about 750, about 800, about 850, about 900, about 950 or about 1000 bases in length, for example from about 100 to about 1000, from about 200 to about 800, from about 500 to about 850, from about 100 to about 500 and from about 300 to about 775 base pairs in length and the like. In specific examples, end joined fragments are selected for sequence determination that are from about 300 to 500 base pairs in length. The term “about” includes embodiments limited to the exact stated length or a length that ±5 bases from the stated lengths.

Treating with Bisulfite

The methods may further comprise treating the nucleic acids (e.g., the end joined nucleic acid fragments) with an agent that modifies the unmethylated base of the nucleic acids. In some embodiments, such treatment (e.g., bisulfite treatment) allows the discrimination between an unmethylated and a methylated base. In some cases, the agent modifies unmethylated cytosine, e.g., the agent alters the chemical composition of unmethylated cytosine but does not change the chemical composition of methylated cytosine. For example, the agent may selectively modify either the methylated or non-methylated form of CpG dinucleotide.

In some examples, the agent that modifies the unmethylated base is sodium bisulfite. Sodium bisulfite comprises sodium hydrogen sulfite having the chemical formula of NaHSO₃. Sodium bisulfite may function to deaminate cytosine into uracil; but does not affect 5-methylcytosine (a methylated form of cytosine with a methyl group attached to carbon 5). When the bisulfite-treated DNA is amplified via polymerase chain reaction, the uracil is amplified as thymine and the methylated cytosine is amplified as cytosine. Suitable chemical reagents include hydrazine and bisulphite ions and the like. In some examples, when treating DNA, sodium bisulfite converts unmethylated cytosine to uracil, while methylated cytosines are maintained. Without wishing to be bound by a theory, it is understood that sodium bisulfite reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonated group can be removed under alkaline conditions, resulting in the formation of uracil. The nucleotide conversion results in a change in the sequence of the original DNA. The resulting uracil has the base pairing behavior of thymine, which differs from cytosine base pairing behavior. To that end, uracil is recognized as a thymine by DNA polymerase. In some cases, after PCR or sequencing, the resultant product contains cytosine only at the position where 5-methylcytosine occurs in the starting template DNA.

In some examples, the treatment (e.g., bisulfite treatment) may be performed prior to nucleic acid isolation (e.g., by capture agents). In some examples, the treatment may be performed prior to any adapter ligation step. In some examples, the treatment may be performed prior to nucleic acid amplification. In some examples, the treatment (e.g., bisulfite treatment) may be performed prior to nucleic acid isolation, adapter ligation, and nucleic acid amplification. In these cases, the negative effects from harsh chemical conditions during the treatment may be avoided in the following nucleic acid isolation, adapter ligation, and nucleic acid amplification steps. In certain examples, it is also contemplated that the treatment step is performed after nucleic acid isolation, adapter ligation, and/or nucleic acid amplification steps.

### Isolating Nucleic Acids

The methods herein may further comprise isolating nucleic acids. In some case, the isolated nucleic acids comprise the bisulfite treated, end joined DNA fragments generated using the methods herein. In some cases, nucleic acid isolation comprises isolating chromatin DNA from other components (e.g., proteins such as histones) in the chromatin. An “isolated” biological component (such as the end joined fragmented nucleic acids described herein) has been substantially separated or purified away from other biological components in the cell of the organism, in which the component naturally occurs, for example, extra-chromatin DNA and RNA, proteins and organelles. Nucleic acids and proteins that have been “isolated” include nucleic acids and proteins purified by standard purification methods, for example, from a sample. It is understood that the term “isolated” does not imply that the biological component is free of trace contamination, and can include nucleic acid molecules that are at least 50% isolated, such as at least 75%, 80%, 90%, 95%, 98%, 99%, or even 100% isolated.

### Capture Agent

In cases where the nucleic acids comprise labeled nucleotides, isolation of such nucleic acids may be performed using the labels. In some examples, the label may be captured by a capture agent. The label may be a chemical reagent or group, protein, enzyme, polysaccharide, oligonucleotide, DNA, RNA, recombinant vector or a small molecule to which the capture agent binds substantially or preferentially. In an example, a capture agent may be capable of binding to a label that is covalently linked to a targeting probe. The label and the capture agent may be a biotin-streptavidin pair, enzymatic moieties may be linked via an ester/amide bond, a thiol addition into a maleimide, Native Chemical Ligation (NCL) techniques, Click Chemistry (i.e. an alkyne-azide pair). For example, the label may be biotin (e.g., for instance by incorporation of biotin-14-CTP or other biotinylated nucleotides) and the capture agent may be streptavidin.

In some cases, the capture agent is a nucleic acid-specific binding agent that binds substantially only to the defined nucleic acid, such as DNA, or to a specific region within the nucleic acid, for example a nucleic acid probe. In certain cases, the capture agent may be a protein-specific binding agent that binds substantially only the defined protein, or to a specific region within the protein. For example, a “specific binding agent” includes antibodies and other agents that bind substantially to a specified polypeptide. Antibodies can be monoclonal or polyclonal antibodies that are specific for the polypeptide, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

In some embodiments the capture agent has been immobilized for example on a solid support, thereby isolating the labeled nucleic acids of interest. By “solid support” is intended any support capable of binding a targeting nucleic acid. Example supports or carriers include glass, polystyrene, polypropylene, polyethylene, dextran, nylon, amylases, natural and modified celluloses, polyacrylamides, agarose, gabbros and magnetite. The nature of the carrier can be either soluble to some extent or insoluble for the purposes of the present disclosure. The support material may have virtually any possible structural configuration so long as the coupled molecule is capable of binding to the targeting probe. Thus, the support configuration may be spherical, as in a bead, or cylindrical, as in the inside surface of a test tube, or the external surface of a rod. Alternatively, the surface may be flat such as a sheet or test strip. In some examples, the solid support may be particles, e.g., beads.

### Fixing Cells

In some embodiments, the methods herein may comprise the optional step of fixing cells. After fixation, the molecules (e.g., nucleic acids) in the cells may be fixed in positions relative to each other. The fixation may be performed by crosslinking. When nucleic acids are cross-linked, either directly or indirectly, the information about spatial relationships between the different nucleic acid fragments in the cell, or cells, is maintained during this joining step herein, and substantially all of the end joined nucleic acid fragments formed at this step were in spatial proximity in the cell prior to the crosslinking step. Therefore, at this point the information about which sequences are in spatial proximity to other sequences in the cell is locked into the end joined fragments. In some cases, the methods comprise holding the nucleic acids in a fixed position relative to one another prior to fragmenting. The nucleic acids may be held in the fixed position by crosslinking the cells or nuclei in the cells or isolated nuclei from the cells.

The fixation may be performed by chemical crosslinking, for example, by contacting the cells or isolated nuclei in the cells with one or more chemical cross linkers. In some embodiments, the cells are fixed, for example with a fixative, such as an aldehyde, for example formaldehyde or glutaraldehyde. In some embodiments, a sample of one or more cells is cross-linked with a cross-linker to maintain the spatial relationships in the cell. For example, a sample of cells can be treated with a cross-linker to lock in the spatial information or relationship about the molecules in the cells, such as the DNA and RNA in the cell.

In some embodiments, the relative positions of the nucleic acid can be maintained without using crosslinking agents. For example, the nucleic acids can be stabilized using spermine and spermidine (see Cullen et al., Science 261, 203 (1993), which is specifically incorporated herein by reference in its entirety). Other methods of maintaining the positional relationships of nucleic acids are known in the art. In some embodiments, nuclei are stabilized by embedding in a polymer such as agarose. In some embodiments, the cross-linker is a reversible cross-linker. In some embodiments, the cross-linker is reversed, for example after the fragments are joined. In specific examples, the nucleic acids are released from the cross-linked three-dimensional matrix by treatment with an agent, such as a proteinase, that degrade the proteinaceous material from the sample, thereby releasing the end ligated nucleic acids for further analysis, such as determination of the nucleic acid sequence. In specific embodiments, the sample is contacted with a proteinase, such as Proteinase K.

In some embodiments of the disclosed methods, the cells are contacted with a crosslinking agent to provide the cross-linked cells. In some examples, the cells are contacted with a protein-nucleic acid crosslinking agent, a nucleic acid-nucleic acid crosslinking agent, a protein-protein crosslinking agent or any combination thereof. By this method, the nucleic acids present in the sample become resistant to special rearrangement and the spatial information about the relative locations of nucleic acids in the cell is maintained. In some examples, a cross-linker is reversible, such that the cross-linked molecules can be easily separated in subsequent steps of the method. In some examples, a cross-linker is a non-reversible cross-linker, such that the cross-linked molecules cannot be easily separated. In some examples, a cross-linker is light, such as UV light. In some examples, a cross linker is light activated.

Examples of cross-linkers include formaldehyde, paraformaldehyde, alcohol (e.g., methanol), disuccinimidyl glutarate, UV light, psoralens and their derivatives such as aminomethyltrioxsalen, glutaraldehyde, ethylene glycol bis[succinimidylsuccinate], bissulfosuccinimidyl suberate, 1-Ethyl-3-[3-dimethylaminopropyl]carbodiimide (EDC) bis[sulfosuccinimidyl] suberate (BS3) and other compounds known to those skilled in the art, including those described in the Thermo Scientific Pierce Crosslinking Technical Handbook, Thermo Scientific (2009) as available on the world wide web at piercenet.com/files/1601673_Crosslink_HB_Intl.pdf, or may involve embedding cells or tissue in a paraffin wax or polyacrylamide support matrix.

Alternatively or additionally, in some situations, it is not necessary to hold the nucleic acids in place using a chemical fixative or crosslinking agent. Thus, in some embodiments, no crosslinking agent is used. In still other embodiments, the nucleic acids are held in position relative to each other by the application of non-crosslinking means, such as by using agar or other polymer to hold the nucleic acids in position.

### Reversing the Crosslinking

In some embodiments, the methods may also comprise reversing the crosslinking at some point. In some examples, the crosslinking may be reversed prior to the nucleic acid shearing, bisulfite treatment, and/or nucleic acid isolation. Reverse crosslinking may be performed by incubating the cells, nuclei, or molecules with detergents (e.g., SDS), proteinase (e.g., proteinase K), and/or at high temperature (e.g., at least 60° C., at least 70° C., at least 80° C., or at least 90° C., such as about 68° C.).

### Cell Lysis and Permeabilization

In some embodiments, the cells are lysed to release the cellular contents, for example after crosslinking. In some cases, the cells are lysed and nuclei are released before nucleic acid fragmentation. In some examples, the nuclei are lysed as well. In other examples, the nuclei are maintained intact, which can then be isolated and optionally lysed, for example using an reagent that selectively targets the nuclei or other separation technique known in the art. In some examples, the sample comprises permeabilized nuclei, multiple nuclei, isolated nuclei, synchronized cells, (such at various points in the cell cycle, for example metaphase) or acellular. In some embodiments, the nucleic acids present in the sample are purified, for example using ethanol precipitation. In example embodiments of the disclosed method, the cells and/or cell nuclei are not subjected to mechanical lysis. In some example embodiments, the sample is not subjected to RNA degradation. In specific embodiments, the sample is not contacted with an exonuclease to remove biotin from un-ligated ends. In some embodiments, the sample is not subjected to phenol/chloroform extraction. In certain embodiments, the cells or nuclei may be permeabilized to allow reagents for processing nucleic acids to contact the nucleic acids.

### Nucleic Acid Shearing

In some embodiments, the end-joined nucleic acid fragments may be sheared to fragments of suitable sizes for further processing. For example, the sheared fragments may have a length from about 100 bp to about 1000 bp, from about 200 bp to about 800 bp, from about 300 bp to about 600 bp, from about 300 bp to about 500 bp, from about 200 bp to about 400 bp, from about 250 bp to about 450 bp, from about 350 bp to about 550 bp, from about 250 bp to about 350 bp, from about 300 bp to about 400 bp, from about 350 bp to about 450 bp, from about 400 bp to about 500 bp, from about 450 bp to about 550 bp, or from about 500 bp to about 600 bp. The term “about” includes embodiments limited to the exact stated length or a length that ±5 bp from the stated lengths.

In some examples, the shearing may be performed by passing the nucleic acid through a narrow capillary or orifice, for example a hypodermic needle, by sonication, such as by ultrasound, by grinding in cell homogenizers, for example stirring in a blender, or by nebulization. In an example, the nucleic acid is sheared by sonication, e.g., using an ultrasonicator.

### Attaching Adapters

The methods may further comprise attaching one or more adapters to the isolated nucleic acids. The adapters may comprise binding sites for primers (e.g., sequence primers, amplification primers, etc.), barcodes, and other elements facilitating nucleic acid analysis and processing. The adapters may be attached to the nucleic acids using ligase or primer extension.

In some cases, the isolated nucleic acids are single stranded DNA. In these cases, one or more adapters may be attached to one end of the single stranded DNA. The adapter(s) may be attached to the 3′ end of the single stranded DNA. In certain cases, the adapter(s) may be attached to the 5′ end of the single stranded DNA. In some cases, both ends of the single stranded DNA may be attached with adapter(s). The adapters may be single stranded.

In some cases, a second strand of DNA may be synthesized using the isolated single stranded DNA, e.g., by primer extension. One or more adapters may be attached to the second strand. The adapter(s) may be attached to the 3′ end of the second strand. In certain cases, the adapter(s) may be attached to the 5′ end of the second strand. In some cases, both ends of the second strand may be attached with adapter(s).

### Amplifying Nucleic Acids

The methods may further comprise amplifying the isolated nucleic acids. In some embodiments, the end joined nucleic acids are selectively amplified. In some examples, to selectively amplify the end joined nucleic acids, a 3′ DNA adaptor and a 5′ RNA adaptor, or conversely a 5′ DNA adaptor and a 3′ RNA adaptor, can be ligated to the ends of the molecules and can be used to mark the end joined nucleic acids. Using primers specific for these adaptors, only end joined nucleic acids may be amplified during an amplification procedure such as PCR. In some embodiments, the target end joined nucleic acid is amplified using primers that specifically hybridize to the adapter nucleic acid sequences present at the 3′ and 5′ ends of the end joined nucleic acids. In some embodiments, the non-ligated ends of the nucleic acids are end repaired. In some embodiments attaching sequencing adapters to the ends of the end ligated nucleic acid fragments. The amplification may be performed with primers with one or more barcodes.

The amplification may be performed using polymerase chain reaction (PCR), quantitative real-time PCR; reverse transcriptase PCR (RT-PCR); real-time PCR (rt PCR); real-time reverse transcriptase PCR (rt RT-PCR); nested PCR; strand displacement amplification; transcription-free isothermal amplification; ligase chain reaction amplification; gap filling ligase chain reaction amplification; coupled ligase detection and PCR; and NASBA™ RNA transcription-free amplification or other methods known in the art.

### Barcodes

The barcodes herein, e.g., those on primers, adapters, or any other nucleic acid molecules, include short sequences of nucleotides (for example, DNA or RNA) used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA, such that multiple species can be sequenced together.

Barcoding may be performed based on any of the compositions or methods disclosed in International Patent Publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

### Determining Sequences

The isolated nucleic acids may be analyzed using various methods, including determining the sequences of the junctions or a portion thereof. The sequence reads may provide physical proximity information of nucleic acids. Such information may be used to determine spatial proximity relationships (e.g., in situ) of the nucleic acids in cells. In some cases, determining the spatial proximity relationships between the nucleic acids comprises identifying chromosomal location of nucleic acid sequences at 5′, 3′ or both 5′ and 3′ of the junctions. Advantageously, the methods allow for simultaneous determining of spatial proximity between nucleic acids and the methylation profile of the nucleic acids.

Alternatively or additionally, the epigenetic profile, e.g., methylation profile, of the junctions or sequences close to the junctions may be determined. In some cases, determining the methylation profile comprises generating a genome-wide methylation profile of cells of interest. The relationship between the spatial proximity and the epigenetic (e.g., methylation) profile of the nucleic acids may be determined. Such relationship may be correlated with a disease, and thus may be used for diagnosing and/or developing a treatment plan for the disease. In some examples, the nucleic acid analysis comprises quantifying a frequency with which pairs of loci in the nucleic acids are found adjacent and/or a frequency with which loci in the nucleic acids are methylated.

### Sequencing

In some embodiments, the methods herein may comprise sequencing the isolated nucleic acids. In some examples, the isolated bisulfite treated and end joined DNA (or a portion thereof) described herein may be sequenced. Sequencing may be used to determine the sequence of and/or adjacent to the junctions. Sequencing may also be used to determine the methylation profile of the DNA. In some embodiments, determining the sequence of a junction includes using a probe that specifically binds to the junction at the site of the two joined nucleic acid fragments. In particular embodiments, the probe specifically hybridizes to the junction both 5′ and 3′ of the site of the join and spans the site of the join. A probe that specifically binds to the junction at the site of the join can be selected based on known interactions, for example in a diagnostic setting where the presence of a particular target junction, or set of target junctions, has been correlated with a particular disease or condition. Once a target junction is known, a probe for that target junction can be synthesized.

Generally, the sequencing can be performed using automated Sanger sequencing (AB13730xl genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®); Moleculo sequencing (see Voskoboynik et al. eLife 2013 2:e00569 and U.S. patent application Ser. No. 13/608,778, filed Sep. 10, 2012); DNA nanoball sequencing; Single molecule real time (SMRT) sequencing; Nanopore DNA sequencing; Sequencing by hybridization; Sequencing with mass spectrometry; and Microfluidic Sanger sequencing. Examples of information that can be obtained from the disclosed methods and the analysis of the results thereof, include without limitation uni- or multiplex, three-dimensional genome mapping, genome assembly, one dimensional genome mapping, the use of single nucleotide polymorphisms to phase genome maps, for example to determine the patterns of chromosome inactivation, such as for analysis of genomic imprinting, the use of specific junctions to determine karyotypes, including, but not limited to, chromosome number alterations (such as unisomies, uniparental disomies, and trisomies), translocations, inversions, duplications, deletions and other chromosomal rearrangements, the use of specific junctions correlated with disease to aid in diagnosis. As would be apparent, forward and reverse sequencing primer sites that are compatible with a selected next generation sequencing platform can be added to the ends of the fragments during the amplification step. In certain embodiments, the fragments may be amplified using PCR primers that hybridize to the tags that have been added to the fragments, where the primer used for PCR have 5′ tails that are compatible with a particular sequencing platform. In certain cases, the primers used may contain a molecular barcode (an “index”) so that different pools can be pooled together before sequencing, and the sequence reads can be traced to a particular sample using the barcode sequence.

In some cases, the sequencing may be next generation sequencing. The terms “next-generation sequencing” or “high-throughput sequencing” refer to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies or single-molecule fluorescence-based method commercialized by Pacific Biosciences. Any method of sequencing known in the art can be used before and after isolation. In certain embodiments, a sequencing library is generated and sequenced. In some examples, the sequencing is performed by transporting the fragments through an orifice in an electric field and measuring change of an electric current density across the orifice when the fragments are transported. The diameter of the orifice may be from 0.1 nm to 10 μm, e.g., from 0.1 nm to 1 nm, 0.5 nm to 5 nm, 1 nm to 10 nm, 10 nm to 100 nm, 100 nm to 1 μm, 1 to 10 μm. Such sequencing method may be a nanopore DNA sequencing method. Examples of nanopore DNA sequencing methods are described in nanoporetech. com/applications/epigenetics.

In some cases, the sequencing may be performed at certain “depth.” The terms “depth” or “coverage” as used herein refers to the number of times a nucleotide is read during the sequencing process. In regards to single cell RNA sequencing, “depth” or “coverage” as used herein refers to the number of mapped reads per cell. Depth in regards to genome sequencing may be calculated from the length of the original genome (G), the number of reads (N), and the average read length(L) as N×L/G. For example, a hypothetical genome with 2,000 base pairs reconstructed from 8 reads with an average length of 500 nucleotides will have 2× redundancy.

In some cases, the sequencing herein may be low-pass sequencing. The terms “low-pass sequencing” or “shallow sequencing” as used herein refers to a wide range of depths greater than or equal to 0.1× up to 1×. Shallow sequencing may also refer to about 5000 reads per cell (e.g., 1,000 to 10,000 reads per cell).

In some cases, the sequencing herein may deep sequencing or ultra-deep sequencing. The term “deep sequencing” as used herein indicates that the total number of reads is many times larger than the length of the sequence under study. The term “deep” as used herein refers to a wide range of depths greater than 1× up to 100×. Deep sequencing may also refer to 100× coverage as compared to shallow sequencing (e.g., 100,000 to 1,000,000 reads per cell). The term “ultra-deep” as used herein refers to higher coverage (>100-fold), which allows for detection of sequence variants in mixed populations.

### Detecting DNA Methylation

In some cases, the DNA methylation may be detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray. Microarrays, and massively parallel sequencing, have enabled the interrogation of cytosine methylation on a genome-wide scale (Zilberman D, Henikoff S. 2007. Genome-wide analysis of DNA methylation patterns. Development 134(22): 3959-3965). Genome wide methods have been described previously (Deng, et al. 2009. Targeted bisulfite sequencing reveals changes in DNA methylation associated with nuclear reprogramming. Nat Biotechnol 27(4): 353-360; Meissner, et al. 2005. Reduced representation bisulfite sequencing for comparative high-resolution DNA methylation analysis. Nucleic Acids Res 33(18): 5868-5877; Down, et al. 2008. A Bayesian deconvolution strategy for immunoprecipitation-based DNA methylome analysis. Nat Biotechnol 26(7): 779-785; Gu et al. 2011. Preparation of reduced representation bisulfite sequencing libraries for genome-scale DNA methylation profiling. Nat Protoc 6(4): 468-481).

In some embodiments, DNA methylation may be detected by whole genome bisulfite sequencing (WGBS) (Cokus, et al. 2008. Shotgun bisulphite sequencing of the Arabidopsis genome reveals DNA methylation patterning. Nature 452(7184): 215-219; Lister, et al. 2009. Human DNA methylomes at base resolution show widespread epigenomic differences. Nature 462(7271): 315-322; Harris, et al. 2010. Comparison of sequencing-based methods to profile DNA methylation and identification of monoallelic epigenetic modifications. Nat Biotechnol 28(10): 1097-1105).

In certain cases, DNA methylation may be detected methylation-specific PCR, whole genome bisulfite sequence, the HELP assay and other methods using methylation-sensitive restriction endonucleases, ChiP-on-chip assays, restriction landmark genomic scanning, COBRA, Ms-SNuPE, methylated DNA immunoprecipitation (MeDip), pyrosequencing of bisulfite treated DNA, molecular break light assay for DNA adenine methyltransferase activity, methyl sensitive Southern blotting, methylCpG binding proteins, mass spectrometry, HPLC, and reduced representation bisulfite sequencing. In some embodiments, the DNA methylation is detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray.

A methylation profile can be determined from the methods disclosed herein. In embodiments, determining the methylation profile comprises generating a genome-wide methylation profile of the cells. Neighborhood methylation profile analysis may be performed by analyzing the loci with which any given locus was in contact. Such analysis may be used to evaluate can how the chromatin neighborhood affected the methylation state of the DNA of that locus. Aggregate methylation profile may also be performed to sum the methylation profile at a large number of positions and to reveal subtle effects in WGBS data. In some examples, aggregate methylation analysis may be performed by plotting DNA methylation in the vicinity of selected sequences (e.g., motifs) and compare it to nucleosome occupancy data (e.g., from MNase-Seq). Methylation profile may comprise unmethylation, methylation and co-methylation at each end of the end-joined nucleic acid fragments.

### Determining a Relationship Between Spatial Proximity and Methylation Profile

In some embodiments, the methods further comprise determining a relationship between spatial proximity and the methylation profile subsequent to determining sequence at the one or more junctions. In some embodiments, the determining of a relationship between spatial proximity and the methylation profile comprises correlating spatial proximity and the methylation profile. For example, the methylation likelihood of a sequence where its neighboring sequence is methylated may be compared with the methylation likelihood of the sequence where its neighboring sequence is not methylated. Such comparison may be used for determining methylation profile of nucleic acid fragments/molecules that are in close spatial proximity. Advantageously, using the method of analysis, Applicants have identified that pairs of sequences that were in spatial proximity had correlated methylation states, regardless of how far apart those sequences lay in the genome by utilization of the method assays disclosed herein.

### Samples

The nucleic acids may be obtained or derived from a sample. A sample, such as a biological sample, may include biological materials (such as nucleic acid and proteins, for example double-stranded nucleic acid binding proteins) obtained from an organism or a part thereof, such as a plant, animal, bacteria, and the like. In particular embodiments, the sample is obtained from an animal subject, such as a human subject. A biological sample may be any solid or fluid sample obtained from, excreted by or secreted by any living organism, including without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood (or fraction(s) or component(s) thereof), plasma, serum, urine, bile, ascites, saliva, cerebrospinal fluid, aqueous or vitreous humor, or any bodily secretion (e.g., mucus, sputum, cervical smear specimens, marrow, feces, sweat, condensed breath, and the like), a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be a sample obtained from any organ or tissue (including a biopsy or autopsy specimen, such as a tumor biopsy) or can include a cell (whether a primary cell or cultured cell) or medium conditioned by any cell, tissue or organ. The samples may be fresh, frozen, preserved in fixative (e.g., alcohol, formaldehyde, paraffin, or PreServeCyte™) or diluted in a buffer. Examples of the samples also include, leaves, stems, roots, seeds, petals, pollen, spore, mushroom caps, and sap.

### Kits

The compositions described herein, such as samples, cells, nucleic acids, and/or other reagents can be supplied in the form of a kit. In such a kit, an appropriate amount the compositions herein may be provided in one or more containers or held on a substrate. The reagents such as nucleic acids, may be provided suspended in an aqueous solution or as a freeze-dried or lyophilized powder, for instance. The container(s) can be any conventional container that is capable of holding the supplied form, for instance, microfuge tubes, ampoules, or bottles. The kits may comprise one or more instructions. The instructions may include directions for obtaining a sample, processing the sample, preparing the probes, and/or contacting each probe with an aliquot of the sample. In certain embodiments, the kit includes an apparatus processing samples, such as individual containers (for example, microtubules) or an array substrate (such as, a 96-well or 384-well microtiter plate). In particular embodiments, the kit includes prepackaged probes, such as probes suspended in suitable medium in individual containers (for example, individually sealed EPPENDORF® tubes) or the wells of an array substrate (for example, a 96-well microtiter plate sealed with a protective plastic film). In some embodiments, kits also may include the reagents necessary to carry out methods disclosed herein. In other particular embodiments, the kit includes equipment, reagents, and instructions for the methods disclosed herein.

### Identifying, Diagnosing and Treating Disease States

The methods described herein may be used for diagnosing a disease or disease state. Characteristics of nucleic acids determined by the methods, such as spatial proximity, epigenetic profile, and their relationships may be compared with reference values in a disease state, wherein a similarity of between one or more of the characteristics and their reference values indicate a disease state. Accordingly, aspects of the disclosed methods relate to diagnosing a disease state based on target junction profile and/or methylation correlated with a disease state, for example cancer, or an infection, such as a viral or bacterial infection. It is understood that a diagnosis of a disease state could be made for any organism, including without limitation plants, and animals, such as humans. In some examples, the methods comprise obtaining cells from the subject; and analyzing nucleic acids in the cells according to the methods herein to determine the spatial proximity and epigenetic (e.g., methylation) profile of the nucleic acids. The spatial proximity and the epigenetic (e.g., methylation) profile are indicative of the disease in the subject.

The present disclosure further provides methods of treating a disease in a subject. In some examples, when the spatial proximity, epigenetic profile, and/or relationships thereof is correlated with a disease state, one or more nucleotides and their mutations response for this correlation may be identified. Such identified nucleotides may be modified to correct the mutations, e.g., by using a CRISPR-Cas system or variants thereof (e.g., a base editing system). In some examples, the methods of treating a disease in a subject comprises obtaining cells from the subject; analyzing nucleic acids in the cells according to the methods described herein; identifying one or more nucleotides in the nucleic acids that are related to the disease based on the spatial proximity relationships and the methylation profile; and modifying at least one of the identified nucleotides.

In some embodiments, simultaneous determination of spatial proximity relationship of nucleic acids and their methylation profile allows for diagnosis or assists in the diagnosis of a disease or condition. In some examples, both the spatial proximity relationship and methylation profiles of one or more genes are compared to reference values. In certain examples, the reference values may be the spatial proximity relationship and methylation profiles of the one or more nucleic acids (e.g., genes) in a diseased cell or tissue. If the comparison shows that both the spatial proximity relationship and methylation profiles are similar to or substantially the same as the reference values, then the comparison result may indicate that the tested subject has the disease. This approach may allow for accurate diagnosis because it uses two parameters (spatial proximity relationship and methylation profile), thus reducing the chance of false positive diagnosis. Alternatively or additionally, the reference values may be the spatial proximity relationship and methylation profiles of the one or more nucleic acids (e.g., genes) in a healthy cell or tissue. If the comparison shows that any one of the spatial proximity relationship and methylation profiles is different (e.g., at a statistically significant level), then the comparison result may indicate that the subject tested has a disease. This approach may allow for accurate diagnosis because it uses two parameters, thus reducing the chance of false negative diagnosis.

In some cases, spatial proximity relationship and methylations profile of one or more genes in diseased cells and healthy cells may be compared. The comparison may be used to identify markers of chromatin architecture and methylation of such disease. The markers may be used as references for diagnosis of the diseases. Large scale screening may be used to investigate the relationship of a test with the control for any particular disease to a subtype of the disease. For example, screening may provide information about diagnosis for each particular type of disease or subtypes of diseases. The criteria may be a cause of the disease or just a biomarker or diagnostics marker if the underlying mechanism of the disease is unknown.

When a subject is diagnosed with a disease based on the comparison of the spatial proximity and/or methylation of one or more nucleic acids with reference values, a treatment of the disease may be administered to correct the spatial proximity and/or methylation of the nucleic acids. In some cases, the treatment may comprise modifying the methylation of a nucleotide. The modification may be based on the methylation profile of its neighboring nucleotide(s). For example, the modification may only be performed on nucleotide(s) whose neighboring nucleotide(s) is also methylated. In certain examples, the modification may only be performed on nucleotide(s) whose neighboring nucleotide(s) is not methylated. Such modification may be performed using enzymes for nucleic acid methylation or demethylation, or regulators (e.g., inhibitors, activators) thereof.

In certain examples, the treatment may comprise editing one or more nucleotides to correct the spatial proximity and/or methylation of the nucleic acids comprising the nucleotide(s). The nucleotides may be edited using one or more programmable nuclease-based editing approach, e.g., those described in Hsu et al., Cell 157, Jun. 5, 2014 1262-1278 for review). Among these, the CRISPR/CAS9 system offers incredible promise (see e.g. Platt et al., Cell 159(2), 440-455 (2014); Shalem et al., Science 3 84-87 (2014); and Le Cong et al., Science 339, 819 (2013)), which are incorporated by reference herein in their entirety. The nucleotides may be edited (e.g., converted to another nucleotide) using base editing technology, e.g., those described in Zhang F. et al., International Patent Publication No. WO 2018213708, Zhang F et al. International Patent Publication No. WO 2019005884, and Kannan et al. International Patent Publication No. WO 2019005886.

### Methods of Screening

The methods of nucleic acid analysis can be utilized for evaluating environmental stress and/or state, for screening of chemical libraries (e.g., drug candidates), and to screen or identify structural, syntenic, genomic, and/or organism and species variations. Aspects of the present disclosure relate to the correlation of an environmental stress or state with the spatial proximity and/or epigenetic profile of the nucleic acids in a sample of cells. For example, a culture of cells, can be exposed to an environmental stress, such as but not limited to heat shock, osmolarity, hypoxia, cold, oxidative stress, radiation, starvation, a chemical (for example, a therapeutic agent or potential therapeutic agent) and the like. After the stress is applied, a representative sample can be subjected to analysis, for example at various time points, and compared to a control, such as a sample from an organism or cell, for example a cell from an organism, or a standard value.

In some embodiments, the disclosed methods can be used to screen chemical libraries (drug candidates) for agents that modulate chromatin architecture, epigenetic profiles, and/or relationships thereof. By exposing cells, or fractions thereof, tissues, or even whole animals, to different members of the chemical libraries, and performing the methods described herein, different members of a chemical library can be screened for their effect on architecture epigenetic profiles, and/or relationships thereof simultaneously in a relatively short amount of time, for example using a high throughput method. The chemicals identified from the screen may be drugs for treating diseases related to the chromatin architecture and epigenetic profiles.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example, the number of amino acids in a polypeptide compound). Millions of chemical compounds can be synthesized through such combinatorial mixing of chemical building blocks.

In some embodiments, the spatial proximity and/or epigenetic profile determined by the disclosed methods may be used to phase polymorphisms and/or assemble individual haplotypes, distinguish between heterozygous and homozygous structural variations, resolve genomic structural genomic variation, including copy number variations, estimate the 1D distance between two fragments of DNA from the same chromosome, assess syntenic relationships between two or more organisms at arbitrary resolution, and/or generate phylogenetic trees and/or ancestral genomes.

The present application also provides aspects and embodiments as set forth in the following numbered Statements:

Statement 1. A method for analyzing nucleic acids in cells, comprising: fragmenting the nucleic acids, the fragmented nucleic acids comprising overhanging ends; filling in the overhanging ends with one or more nucleotides comprising a label; joining the filled in ends to create one or more end joined nucleic acid fragments with one or more junctions; treating the end-joined nucleic acid fragments with bisulfite; isolating the bisulfite-treated, end-joined nucleic acid fragments using the label; and determining sequence at the one or more junctions in the bisulfite treated end-joined nucleic acid fragments, thereby determining spatial proximity between the nucleic acids and the methylation profile of the nucleic acids.

Statement 2. The method of Statement 1, further comprising determining a relationship between the spatial proximity and the methylation profile.

Statement 3. The method of any one of Statements 1-2, further comprising holding the nucleic acids in a fixed position relative to one another prior to fragmenting.

Statement 4. The method of any one of Statements 1-3, wherein the nucleic acids are held in the fixed position by crosslinking the cells or nuclei in the cells.

Statement 5. The method of any one of Statements 1-4, further comprising reversing the crosslinking.

Statement 6. The method of any one of Statements 1-5, further comprising isolating nuclei from the cells prior to fragmenting.

Statement 7. The method of any one of Statements 1-6, further comprising permeabilizing the nuclei.

Statement 8. The method of any one of Statements 1-7, wherein the nucleic acids are a part of chromatin.

Statement 9. The method of any one of Statements 1-8, wherein the nucleic acids are DNA.

Statement 10. The method of any one of Statements 1-9, wherein fragmenting is performed by digesting the nucleic acids using a nuclease.

Statement 11. The method of any one of Statements 1-10, wherein the nuclease is methylation insensitive.

Statement 12. The method of any one of Statements 1-11, further comprising, prior to the bisulfite treatment, shearing the nucleic acids.

Statement 13. The method of any one of Statements 1-12, wherein the sheared nucleic acids have a length from about 300 base pairs (bp) to about 500 bp.

Statement 14. The method of any one of Statements 1-13, wherein the bisulfite treated, end-joined nucleic acid fragments are isolated using a capture agent that binds to the labeled nucleotides.

Statement 15. The method of any one of Statements 1-14, wherein the capture agent is attached to a solid support.

Statement 16. The method of any one of Statements 1-15, wherein the solid support is a bead.

Statement 17. The method of any one of Statements 1-16, further comprising attaching one or more adaptors to the bisulfite treated end-joined nucleic acid fragments.

Statement 18. The method of any one of Statements 1-17, wherein the one or more adaptors are attached after isolating the bisulfite treated, end-joined nucleic acid fragments.

Statement 19. The method of any one of Statements 1-18, further comprising amplifying the bisulfite treated, end-joined nucleic acid fragments.

Statement 20. The method of any one of Statements 1-19, the bisulfite treated, end-joined nucleic acid fragments are amplified using primers with one or more barcodes.

Statement 21. The method of any one of Statements 1-20, further comprising quantifying: a frequency with which pairs of loci in the nucleic acids are found adjacent, and a frequency with which loci in the nucleic acids are methylated.

Statement 22. The method of any one of Statements 1-21, wherein determining the spatial proximity between the nucleic acids comprises identifying chromosomal location of nucleic acid sequences both 5′ and 3′ of the junctions.

Statement 23. The method of any one of Statements 1-22, wherein determining the methylation profile comprises generating a genome-wide methylation profile of the cells.

Statement 24. The method of any one of Statements 1-23, further comprising correlating a relationship between the spatial proximity and the methylation profile with a disease.

Statement 25. The method of any one of Statements 1-24, wherein the sequence at one or more junctions in the bisulfite treated, end-joined nucleic acid fragments is determined by transporting the fragments through an orifice in an electric field and measuring change of an electric current density across the orifice when the fragments are transported.

Statement 26. The method of any one of Statements 1-25, wherein the sequence at the one or more junctions in the bisulfite treated, end-joined nucleic acid fragments is determined by nanopore sequencing.

Statement 27. A method of diagnosing a disease in a subject, comprising: obtaining cells from the subject; analyzing nucleic acids in the cells according to the method of claim 1, wherein the spatial proximity and the methylation profile are indicative of the disease in the subject.

Statement 28. A method of treating a disease in a subject, comprising: determining spatial proximity and methylation profile of a gene in a cell from the subject; comparing the spatial proximity and the methylation profile to reference values, thereby identifying one or more nucleotides in the gene related to the disease; and modifying at least one of the identified nucleotides.

Statement 29. The method of Statement 28, wherein the spatial proximity and the methylation profile of the one or more identified nucleotides are indicative of the disease.

Statement 30. The method of any one of Statements 28-29, wherein modifying at least one of the identified nucleotides comprises modifying methylation of the at least one of the identified nucleotides.

Statement 31. The method of any one of Statements 28-30, wherein modifying at least one of the identified nucleotides comprises converting at least one of the identified nucleotides to another nucleotide.

Statement 32. A method for screening chemical libraries for agent modulating chromatin architecture and epigenetic profiles, comprising: exposing cells to members of the chemical libraries; determining the spatial proximity and methylation profile of according to Statement 1; and comparing the spatial proximity and the methylation profile to spatial proximity and methylation profile of control cells, thereby identifying members in the chemical libraries that have effects on the spatial proximity and methylation profile.

The invention is further described in the following examples, which do not limit the scope of the invention described in the claims.

### EXAMPLES

### Example 1

This example shows an example method (Hi-Culfite assay) for determining relationships between chromatin contacts and DNA methylation state. Hi-Culfite, a protocol combining Hi-C and whole-genome bisulfate sequencing (WGBS), determined chromatin contacts and DNA methylation simultaneously. Hi-Culfite also revealed relationships that cannot be seen when the two assays are performed separately. For instance, Applicants show that loci associated with open chromatin exhibited context-sensitive methylation: when their spatial neighbors lay in closed chromatin, they were much more likely to be methylated.

Nuclear architecture and DNA methylation play critical and interdependent roles in mammalian genome regulation (1,2). For example, the insulator protein CTCF, which interacts with the cohesin complex to form chromatin loops and thereby establish discrete structural and functional segments of the genome, binds in a methylation-sensitive fashion (3,4). Similarly, a recent study has implicated hyper-methylation induced disruption of chromosome topology in oncogene activation (5).

In previous studies, nuclear architecture and DNA methylation were interrogated independently using Hi-C proximity ligation mapping6 and whole-genome bisulfite sequencing (WGBS) (7), respectively. However, independent assays of architecture and methylation can obscure the interdependence of the underlying phenomena. In addition, since both sequencing assays require deep coverage of the genome, performing them separately increases the cost.

Applicants have developed Hi-Culfite, a combined protocol integrating in situ Hi-C (8) and bisulfite conversion (9). Applicants show that Hi-Culfite generated chromatin contact and DNA methylation maps. Hi-Culfite data sets also allowed integrated, multi-omics analyses that revealed unique biological insights, such as relationships between DNA methylation and spatial context, which cannot be obtained from separate Hi-C and WGBS data sets.

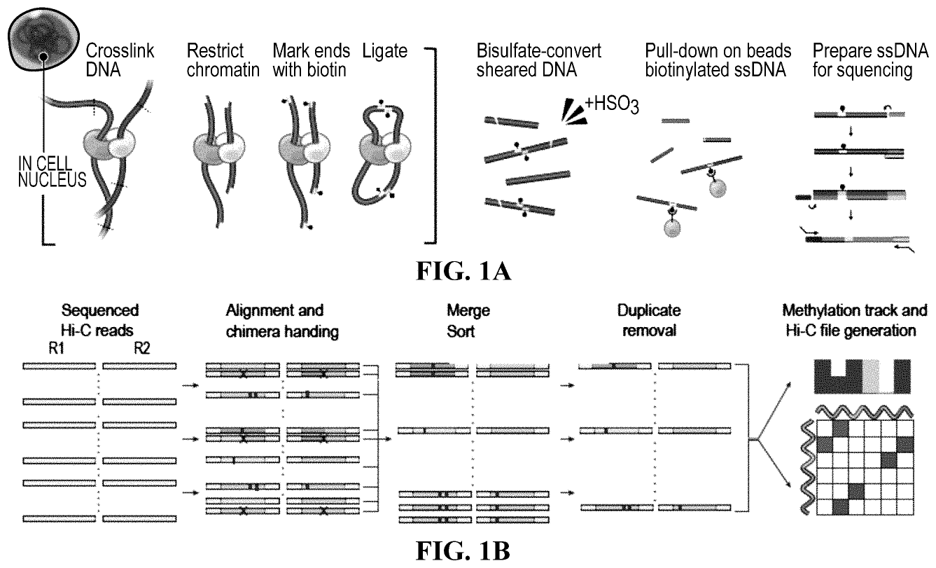

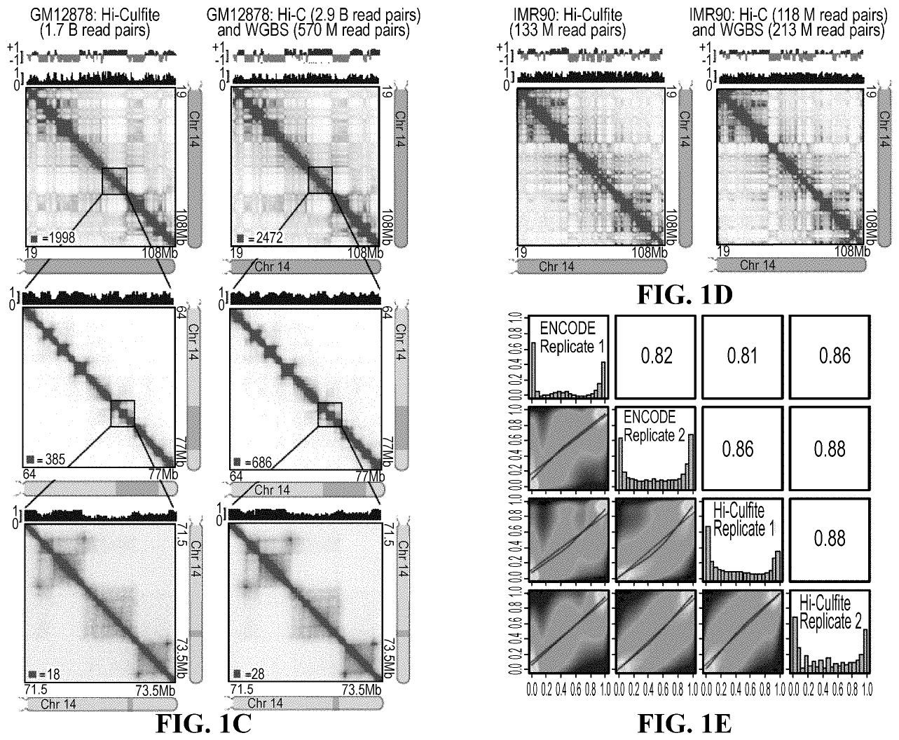

Hi-Culfite library construction (FIG. 1A) followed the initial steps of an in situ Hi-C protocol (8). One million cells were crosslinked with formaldehyde; chromatin was digested with a restriction enzyme; and the overhangs were filled in, incorporating a biotinylated nucleotide to mark the fragment ends after ligation. The DNA was then purified and sheared. Next, Applicants performed bisulfite conversion, which converted unmethylated cytosine residues into uracil but left methylated cytosine residues intact. Applicants captured the resulting single-stranded, biotin-marked ligation products on streptavidin beads. Finally, Applicants added adapters to the 3′ end, synthesized the second strand by primer extension, ligated adapters to the 3′ end of the 2nd strand, and PCR amplified with indexed primers.

Applicants found that by performing bisulfite conversion prior to biotin pulldown and adapter ligation, Applicants achieved much higher yield. By contrast, early iterations of the protocol, in which bisulfite conversion was performed on un-amplified Hi-C libraries with methylated adapters, led to great drops in library complexity (i.e., the number of unique sequencing templates in the library). This was presumably due to extensive strand breakage on account of the harsh chemical conditions during the bisulfite conversion, leading to a reduction of the number of PCR-amplifiable molecules (10). For instance, at the final amplification step, 10 cycles of PCR resulted in Hi-Culfite libraries with a mean concentration of 32 nM, comparable to Applicants' standard in situ Hi-C protocol 8. By contrast, the “adapter first” workflow produced ˜100 times less library (0.2 nM) despite starting with 10-fold more cells (10 million) and two additional PCR cycles.

A Hi-Culfite map comprised pairs of neighboring bisulfate-converted DNA sequence reads, each indicating the methylation state of two loci that might lie far apart along the genome, but that were spatially adjacent at the time of the assay. By quantifying the frequency with which pairs of loci were found adjacent, a Hi-Culfite map, such as a Hi-C map, was used to create a contact matrix showing the frequency at which pairs of loci co-localize. By quantifying how often loci were methylated, the Hi-Culfite map was used to create a genome-wide methylation profile.

Applicants sought to validate the quality of Hi-Culfite results by comparing contact matrices and methylation profiles generated using Hi-Culfite to those produced when in situ Hi-C and WGBS experiments were performed separately. To do so, Applicants performed a Hi-Culfite experiment in GM12878 lymphoblastoid cells, for which both Hi-C8 and WGBS data sets (DCC accession: ENCSR890UQO11,12) were publicly available. After initial quality control of the Hi-Culfite libraries, Applicants generated a deep Hi-Culfite dataset containing a total of 1.75 billion read pairs (Table 1), comparable to the ENCODE standard for in situ Hi-C (2 billion).

Applicants found that the chromatin contact maps produced by Hi-Culfite were comparable to in situ Hi-C maps across all resolutions that were examined (1 Mb->5 kb; FIG. 1C). Compartments (which arose when open [“compartment A”] and closed [“B”] chromatin segregate in the nucleus, manifesting as a plaid pattern in the contact map), contact domains (intervals in which all loci exhibited an enhanced contact frequency within themselves, manifesting as a bright square along the diagonal of the contact map), and loops (pairs of loci exhibiting an enhanced contact frequency with one another relative to their 1D genomic neighborhood, manifesting as peaks in the contact map) were evident in the Hi-Culfite data. Similarly, when Applicants compared their results to ENCODE WGBS data, Applicants found that the methylation profiles closely resembled one another at all resolutions. Indeed, the average single-CpG methylation correlation between Hi-Culfite and ENCODE experiments of r=0.85 (range 0.81-0.88) exceeded the minimum r≥0.8 standard defined by ENCODE for replicate WGBS experiments (FIG. 1E).

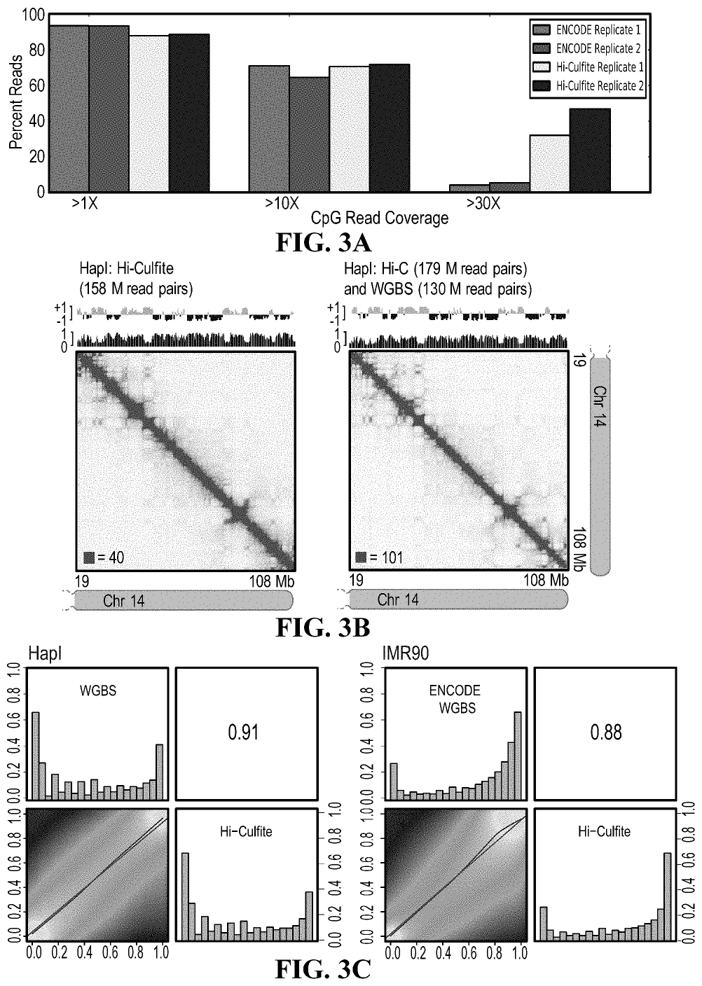

Because an in situ Hi-C data set at loop resolution might need more reads than a typical WGBS experiment (as reflected in, for instance, the ENCODE standards for the two protocols), the methylation track emerging from the loop resolution Hi-Culfite experiment in GM12878 had much deeper coverage than the corresponding ENCODE WGBS experiments (84× as compared to the ENCODE standard of 30×). Despite the presence of a larger number of reads, the Hi-Culfite protocol described here covered slightly fewer CpG sites than WGBS: 88.3% versus 90.4% covered by at least one read (FIGS. 3A-3C). This was because CpGs far from a restriction site—typically those in low-complexity regions of the genome—were rarely covered using the Hi-Culfite protocol. However, when Applicants examined CpGs that were covered multiple times in WGBS experiments, this effect disappeared. For instance, 71.2% of CpGs were covered at least 10-fold in Applicants' Hi-Culfite experiment, versus 67.9% for ENCODE WGBS; and 39.5% of CpGs were covered 30-fold or higher in Applicants' Hi-Culfite experiment, versus 4.8% for ENCODE WGBS.

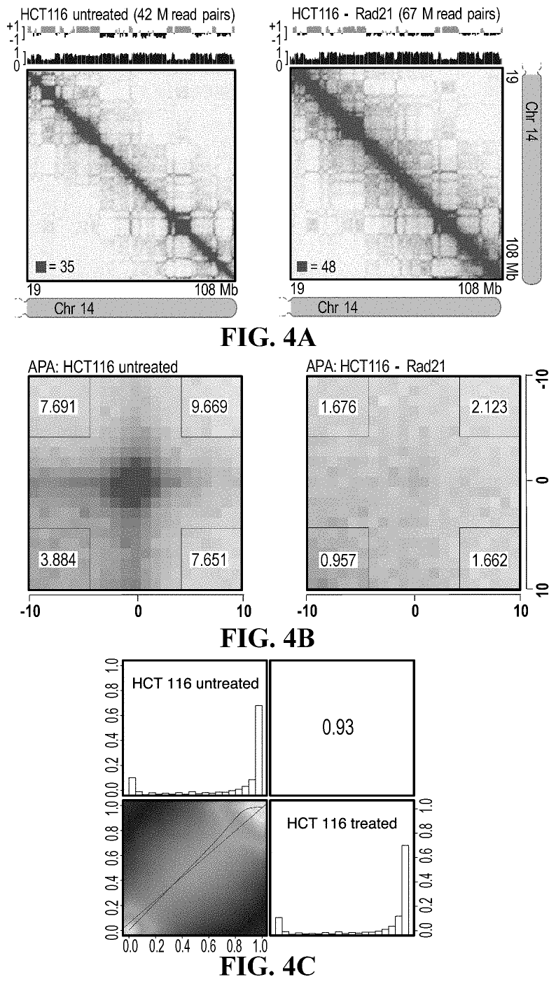

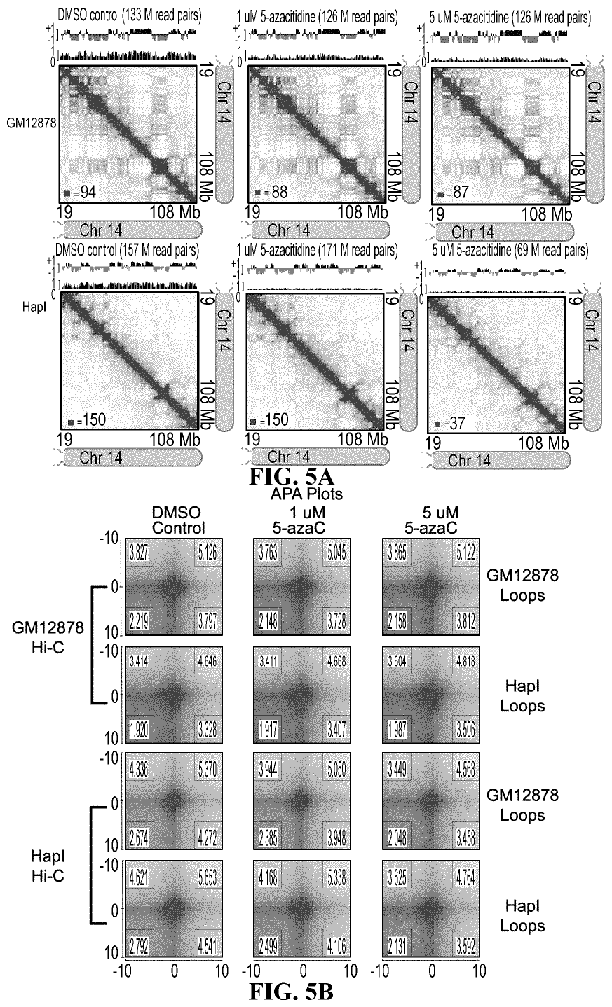

When the above analyses were repeated in other cell lines, including IMR90 (lung myofibroblasts) and HAP1 (haploid chronic myelogenous leukemia), Applicants obtained similar results (FIG. 1D, FIGS. 3A-3C). Applicants also confirmed that Hi-Culfite could recapitulate the effects of well-characterized perturbations to nuclear architecture and DNA methylation. For instance, 5-azacytidine is known to inhibit DNA methylation (13). When Applicants performed Hi-Culfite on GM12878 and on HapI cells treated with 5-azacytidine, Applicants found that methylation declined genome-wide, as expected (on average by 2-fold see FIGS. 4A-4C, Table 2). Applicants did not discern an impact on nuclear architecture (FIGS. 4A-4C). Degradation of cohesin is known to lead to the disappearance of CTCF-mediated looping (15,16,17). When Applicants degraded cohesin in HCT-116 colorectal carcinoma cells (Online Methods) and assayed the results using Hi-Culfite, Applicants found that chromatin loops disappear, as expected (FIGS. 5A-5B). By contrast, Applicants observed no effect on WGBS (FIGS. 5A-5B). Note that Applicants used shallower maps for these analyses, ranging from 42 M to 171 M read pairs.

As shown herein, Hi-Culfite made it possible not only to generate both the contact map and methylation profile data at once, but to perform integrative analysis of the underlying phenomena in ways that were not feasible when the assays were performed separately. In Hi-Culfite, each bisulfite-transformed read's neighbor sequence provided additional information about its chromatin neighborhood. Since the vast majority (typically 75%) of ligations in an in situ Hi-C experiment happened in cis8, and nearly all of them happened in the same nucleus (8,18,19), Hi-Culfite could provide insights about long-range epigenetic concordance and co-regulation that were not visible in ensemble DNA methylation measurements on heterogeneous cell populations.

For instance, to determine the effect of chromatin neighborhood on the methylation state of DNA, Applicants partitioned the genome into loci of 500 kb each. Applicants then calculated how often sequences derived from an index locus were methylated (i.e., exhibited mostly methylated CpGs) conditioned on the identity of the locus from which their neighbor sequence originated. Strikingly, Applicants found that the methylation frequency of a given sequence was strongly associated with this spatial context. As an example, sequences deriving from the locus chr14: 37-37.5 Mb were methylated 64% of the time when the neighbor was locus chr14: 68.5-69 Mb (in 14 out of 22 cases), but only 6% of the time when the neighbor was locus chr14: 75.5-76 Mb (in 1 out of 17 cases).

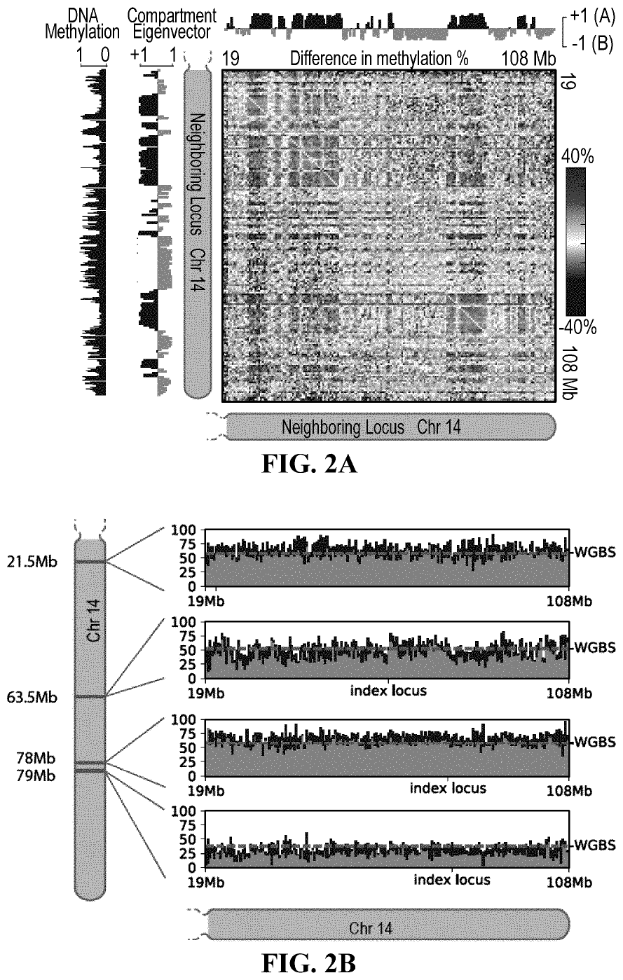

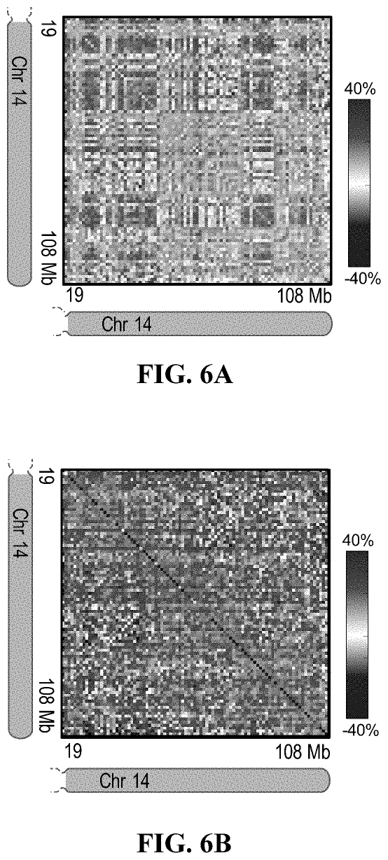

Applicants therefore generated a matrix showing the mean methylation frequency of every locus (i.e., what fraction of the time sequences from the locus were methylated) as a function of the identity of the neighboring locus. This revealed that the methylation frequency of reads derived from the A compartment depended especially strongly on their spatial context. When loci in the A compartment (open chromatin) had a neighbor in the B compartment (closed chromatin), they were methylated 34% more often than when their neighbor was in the A compartment. By contrast, loci in the B compartment exhibited less dependence on spatial context: when their neighbor was in the A compartment, they were methylated 7% less often than when their neighbor was in the B compartment (FIG. 2A, FIGS. 6A-6B).

Next, Applicants asked whether the methylation state of a read tended to correlate with the methylation state of the neighboring sequence. Applicants found that, regardless of the identity of the neighboring locus or how far away it lay along the contour of the chromosome, the likelihood that a read was methylated was higher, increasing from 40% to 52% if the neighboring read was also methylated (FIG. 2B). This demonstrated that pairs of sequences that were in spatial proximity had correlated methylation states, regardless of how far apart those sequences lay in the genome.

Finally, to facilitate multi-omics analysis of Hi-Culfite data, Applicants enhanced the Juicer software for Hi-C data analysis (20). The new module, dubbed JuiceMe (github.com/aidenlab/juiceme), was designed to work with Hi-Culfite data, allowing users to create whole-genome contact maps, whole-genome bisulfite tracks, and quality control statistics relevant to both data types (FIG. 1B; Table 1). In addition, Applicants enhanced the Juicebox visualization system21 to enable interactive exploration of Hi-Culfite data (github.com/aidenlab/juicebox).

Taken together, this example shows a new set of experimental and computational tools for simultaneously probing both the nucleome and the methylome. The results demonstrated that the methylation state of a sequence was context-sensitive, depending not only on the loci in its spatial neighborhood, but also on their methylation state. More generally, the results highlighted the ways in which Hi-Culfite, and other multi-omics approaches, enabled Applicants to integrate disparate data types into new insights about the interplay between cellular phenomena.

### Materials and Methods

### Hi-Culfite Protocol

Cell Culture: All commercial cell lines were cultured following vendors' recommendations to about 80% confluency before they were harvested and cross-linked in fresh complete medium. For methylation inhibition experiments, cultures of GM12878 (Coriell Institute) and Hap1 (Horizon) were grown for 8 days in medium supplemented with DMSO without drug or with 1 μM or 5 μM 5-azacytidine (Sigma Aldrich) solubilized in DMSO, refreshing the medium/drug every two days.

IMR90 (CCL-186) cell line was purchased from ATCC and expanded as recommended.

Applicants obtained HCT-116-CMV-OsTir1 and HCT-116-RAD21-mAID-mClover cells (HCT-116 RAD21-mAC) from Masato Kanemaki (24). The cells were cultured in McCoy's 5A medium supplemented with 10% FBS, 2 mM L-glutamine, 100 U/ml penicillin, and 100 μg/ml streptomycin at 37° C. with 5% CO2. Degradation of the AID-tagged RAD21 was induced by the addition of 500 μM indole-3-acetic acid (IAA; Sigma Aldrich). For experiments with untreated cells and cells treated for 6 hours, medium was aspirated at t=0, and either replaced with fresh medium (untreated) or medium containing 500 μM IAA. The cells were then washed, trypsinized and processed for downstream experiments at t=6 hrs.

Library Construction: Hi-C libraries were prepared using the protocol described (8) Briefly, one million cells were crosslinked with 1% formaldehyde for 10 minutes at room temperature and then quenched with 0.2 M glycine solution. Cells were lysed and nuclei permeabilized with 0.5% SDS for 10 minutes at 62° C. Chromatin was digested with 100 U of MboI restriction enzyme (NEB). Ends of the restriction fragments were filled-in and labeled with a biotinylated nucleotide and then ligated. Nuclei were pelleted, proteins were digested with proteinase K, and crosslinks were reversed by heating at 68° C. overnight. DNA was sheared in a Covaris focused ultrasonicator to a length of 300-500 bp. Size-selected DNA was split for processing with two workflows—10% of the material was used for preparation of a regular Hi-C library (unconverted control) and 90% of the DNA was used for Hi-Culfite library construction. Hi-C libraries were finished by enriching for biotinylated ligation junctions through binding to T1 streptavidin beads (Thermo Fisher) and preparing the library for Illumina sequencing performing the end-repair, A-tailing and adapter ligation steps with DNA attached to the beads. Libraries were amplified directly off the beads and purified for subsequent Illumina sequencing.

DNA for Hi-Culfite was first treated with sodium bisulfite using EpiTect Fast bisulfite conversion kit (Qiagen) following the kit's instructions and extending each of the two 60° C. conversion incubations to 20 minutes. Converted DNA was purified without addition of an RNA carrier to the binding buffer. Biotinylated ligation junctions in purified bisulfite-converted DNA were captured on 15 μl C1 streptavidin beads (Thermo Fisher) in denaturing binding buffer (10 mM Tris pH 7.5; 5 mM EDTA; 500 mM LiCl; 0.5% Igepal CA630; 0.2% SDS; 4 M Urea) for 10 minutes at 55° C. DNA was washed twice with binding buffer at 55° C. and once with 10 mM Tris. Beads were resuspended in 15 μl of 10 mM Tris, and libraries were detached from the beads by heating for 5 minutes at 95° C. After separating on a magnet, library construction for Illumina sequencing was performed on the supernatant containing ssDNA library using Accel-NGS Methyl-Seq kit (Swift Biosciences) following the kit's manual.

Libraries were amplified with barcoded primers using 8-10 amplification cycles. Final libraries were purified and molecules in the range of 450-650 bp were selected by agarose gel electrophoresis and subsequent gel extraction.

Both Hi-C and Hi-Culfite libraries were first sequenced with 80 bp paired-end reads on an Illumina NextSeq instrument, obtaining about 2 million read pairs per library. Data quality was evaluated and successful libraries were then deep sequenced with 150 bp paired-end reads on the Illumina HiseqX platform.

### JuiceME: Hi-Culfite Data Processing Pipeline

The data processing pipeline for Hi-Culfite was a modified version of the Juicer pipeline (20). Since the DNA has been bisulfite-converted, the aligner might be able to handle mapping to essentially two different genomes. Additionally, after alignment the reads might be combined to generate WGBS sequencing tracks. The other steps of the Juicer pipeline (chimera handling, duplicate removal, Hi-C contact map creation and normalization) remained the same.

Sequence Alignment with bwa-meth: All Hi-Culfite data reported in this example was generated using Illumina paired-end sequencing. The sequencer produces two fastq files, one for each read end. As with any proximity ligation assay, each read end might be aligned separately as a single end read so that the aligner did not make incorrect assumptions about the insert size.

Applicants used bwa-meth22 as the aligner. Bwa-meth used bwa25 as its base aligner, which performed well on Hi-C data20. Bwa-meth worked by creating an alternate methylated version of the genome and then calling bwa to align. Since each read end was aligned separately, Applicants first reversed complement the second read end before calling bwa-meth. All reads that align were merged into a BAM file that was coordinate-sorted for methylation processing. For the Hi-C contact map creation, the rest of the pipeline proceeded exactly as previously described (20): chimeras were appropriately handled, duplicates and near duplicates were removed, and contact maps were created and normalized.

Methylation Track Generation with MethylDackel: Duplicate Hi-C contacts were marked as duplicates in the methylation BAM. Applicants then called the program Methyl Dackel extract with the flag “-F 1024”; this ignored duplicates reads but kept all other mapped reads with MAPQ ≥10. MethylDackel generated CpG methylation tracks, a cytosine coverage report, and an input file for the analysis program MethylKit26. Applicants used MethylKit to produce the correlation analysis in FIGS. 1A-1E. The CpG methylation tracks were bedGraph; Applicants used igvtools27 to create a TDF file for fast viewing.

Applicants called MethylDackel perRead to produce methylation information per read instead of the usual per cytosine. The results from this program were then combined with the list of Hi-C contacts in order to create binned contact maps separated by methylation status. Each contact that had methylation status information on both read ends was classified as either “both methylated” (both read ends are methylated), “both unmethylated” (both read ends are unmethylated), or “methylated-unmethylated” (one read end was methylated and the other was unmethylated). These contact maps were used in the co-methylation analysis, described below.

### Neighborhood Methylation Analysis

For the neighborhood methylation analysis, Applicants determined how the chromatin neighborhood (i.e., the loci that any given locus was in contact with) affected the methylation state of the DNA of that locus. That is, Applicants wanted to know the methylation percentage of locus i given that it interacts with locus j. Each Hi-C contact in this analysis had a methylation status of 0 or 1 on each read end, based on whether or not the methylation status of the CpGs it covered result in >50% methylation. Applicants then split Hi-C contacts into four different matrices: contacts in which both read ends were methylated, contacts in which both read ends were unmethylated, contacts in which read end i was methylated and read end j was unmethylated, and contacts in which read end i was unmethylated and read end j was methylated. The latter two matrices were transposes of one another, so Applicants defined these matrices as, respectively, M, U Y, and Y^{T}.

Then the probability that locus i was methylated given that it is in contact with locus j was the sum of contacts at locus i,j in which locus i was methylated, divided by the total number of contacts at locus i,j.

O(i,j)=(M(i,j)+Y(i,j))/M(i,j)+Y(i,j)+Y^{T}(i,j)+U(i,j))

Now consider the null hypothesis. If there were no effect of neighborhood on methylation status, Applicants would expect the methylation percentage of locus i given that it interacted with locus j to be the same regardless of locus j. Define a as the one-dimensional average methylation at locus i:

a(i)=ΣⱼO(i,j)

Then

E(i,j)=a(i) for all j

The matrix in FIG. 2A is the O minus E; its entries gave a measure of methylation frequency divergence from the expected model. Where O-E was O, the data was compatible with the null model, i.e. solely based on the overall average methylation at that locus. High or low values indicated a divergence whereby locus i was more or less methylated than would expected due to its interaction with locus j.

### Methylation Correlation Analysis

For the methylation correlation analysis, Applicants set to determine if the methylation state of a read correlates with the methylation state of the neighboring sequence. Applicants defined the methylation correlation as the frequency with which locus j was methylated given that locus i was methylated, divided by the total number of times locus j was methylated. This was

M(i,j)/(M(i,j)+YT(i,j)

Similarly, Applicants examined the unmethylation correlation: the number of times j was unmethylated given that i is unmethylated, divided by the total number of times locus j is unmethylated. This is

U(i,j)/(I(i,j)+Y(i,j))

If methylation status were uncorrelated with the methylation state of the neighboring sequence, rows of these correlation matrices would simply equal the average methylation status at locus i, i.e. a(i). As shown in FIG. 2B, this was not the case; the methylation likelihood changed when the neighboring read was methylated.

### Supplemental Co-Methylation Analysis

For the comethylation analysis, Applicants set to determine if Hi-C contacts had both ends methylated or both ends unmethylated at a higher frequency than one would expect given the baseline methylation of the loci. Using the matrices defined above, the observed comethylation frequency was

(M(i,j)+U(i,j))/(M(i,j)+Y(i,j)+YT(i,j)+U(i,j))

The expected comethylation frequency was calculated from the average methylation vector a. It was the probability that both were methylated plus the probability that both were unmethylated:

a(i)*a(j)+(1−a(i))*(1−a(j))

In FIGS. 6A-6B, Applicants showed the observed comethylation frequency minus the expected comethylation frequency. The matrix was mostly positive, indicating that loci were comethylated more often than the null hypothesis would indicate.

### Supplemental Aggregate Methylation Analysis

GM12878 CTCF peak calls from ENCODE11,12 were intersected to find common CTCF peaks. This set of peaks was intersected with the HG19 CTCF motif database hosted for Juicer (20), which was originally built using FIM028. CTCF motifs peaks were split into forward and reverse motifs. Forward and reverse CTCF motif peaks were further subdivided into looping and non-looping motifs, by their presence or absence in the GM12878 loop list with motifs8.

Methylation data was generated using the JuiceMe pipeline for the respective Hi-C experiments. Bedgraph files were converted to bigwig files using UCSC executables (29). Aggregation analysis using the CTCF motif peaks and methylation data was performed using bwtool30, and post-processed and visualized using python code hosted at github.com/aidenlab/JuiceMe.

### Supplemental Aggregate Peak Analysis (APA)

APA was performed using Juicer Tools (3) on the Hi-C maps at 25 kb (unless otherwise specified), using loop lists for the maps generated from prior experiments. Loop lists for GM12878, HapI, and HCT-116 were previously published (8,15,31).

### REFERENCES

1. Jenkinson, G., Pujadas, E., Goutsias, J. & Feinberg, A P. Nat. Genet. 49, 719-729.
2. Raineri E, et al. Preprint at .biorxiv.org/content/early/2018/08/04/384578 (2018)
3. Bell, A. C. & Felsenfeld G. Nature 405, 482-485 (2000).
4. Hark A T, et al. Nature 405, 486-9 (2000).
5. Flavahan W A, et al.. Nature 529, 110-4 (2016).
6. Lieberman-Aiden E, et al. Science 326, 289-93(2009).
7. Lister R, et al. Nature 462, 315-22 (2009).
8. Rao S S P, et al. Cell 159, 1665-80 (2014).
9. Frommer M, et al. Proc Natl Acad Sci USA 89, 1827-31 (1992).
10. Miura F, Enomoto Y, Dairiki R, Ito T. Nucleic Acids Res. 40, e136 (2012).
11. ENCODE Project Consortium. Nature 489, 57-74 (2012)
12. Sloan C A, et al. Nucleic Acids Res. 44, D726-32 (2016).
13. Christman J K. Oncogene 21, 5483-95 (2002)
14. Nothjunge S, et al. Nat Commun. 8, 1667 (2017).
15. Rao S S P, et al. Cell 171, 305-320 (2017).
16. Schwarzer W, et al. Nature 551, 51-56 (2017).
17. Wutz G, et al. EMBO J. 36, 3573-3599 (2017).
18. Nagano T, et al. Nature 502, 59-64 (2013).
19. Gavrilov A A, et al. Nucleic Acids Res. 41, 3563-75 (2013).
20. Durand N C, et al. Cell Syst. 3, 95-8 (2016).
21. Durand N C, et al. Cell Syst. 3, 99-101 (2016).
22. Pedersen B S, et al. Preprint at arxiv.org/abs/1401.1129 (2014).
23. Akalin A, et al. Genome Biol. 13, R87 (2012).
24. Natsume T, Kiyomitsu T, Saga Y, Kanemaki M T. Cell Rep. 15, 210-218 (2016).
25. Heng Li. arXiv:1303.3997v2 [q-bio.GN] (2013).
26. Akalin A, et al. Genome Biol. 13, R87 (2012).
27. Thorvaldsdóttir H, Robinson J T, Mesirov J P. Brief Bioinform. 14, 178-92 (2012).
28. Grant C E, Bailey T L, Noble W S. Bioinformatics. 27, 1017-8 (2011)
29. Kent W J, et al. D. Genome Res. 12, 996-1006 (2002).
30. Pohl A, Beato M. Bioinformatics. 30, 1618-9 (2014).
31. Sanborn A L, et al. Proc Natl Acad Sci USA. 112 E6456-65 (2015)

### Additional Methods and Materials

### Aggregate Methylation Analysis

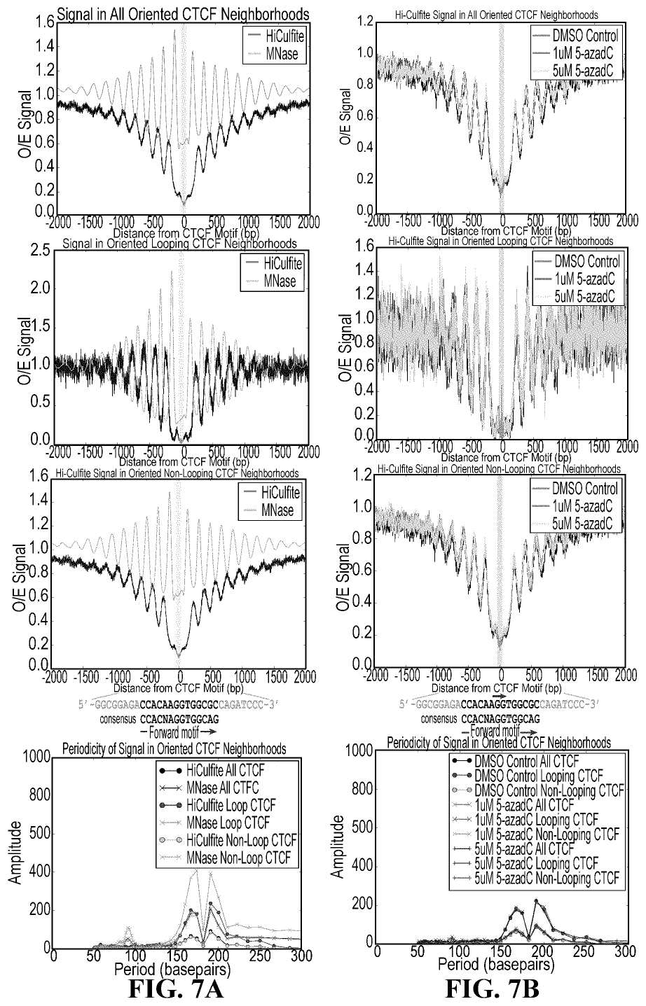

Subtle effects in WGBS data were revealed only when studied in aggregate (i.e., summing the methylation profile at a large number of positions), but were not clearly visible when loci were examined one at a time. Having confirmed that the methylation profiles produced by Hi-Culfite data match WGBS-derived profiles across loci and resolutions, Applicants wondered whether Hi-Culfite data would recapitulate these aggregate effects as well. For example, aggregate analyses of WGBS data had shown that DNA methylation was depleted at CTCF-bound sites (1) and negatively correlated with nucleosome occupancy in the flanking regions (2, 3). To confirm that this relationship is accurately observed using Hi-Culfite, Applicants plotted DNA methylation in the vicinity of CTCF motifs and compared it to nucleosome occupancy data from MNase-Seq (4, 5) (DCC accession ENCSR000CXP). Indeed, Applicants observed the DNA methylation was greatly depleted at CTCF-bound sites, and that the flanking regions exhibited peaks and troughs in methylation that were exactly out of phase with the position of nucleosomes (FIGS. 7A-7B).

Moreover, loops form between CTCF-bound anchor sites in the convergent orientation, i.e., with the motifs pointing towards one another (6). Comparing DNA methylation on opposite sides of a CTCF-bound motif, Applicants found that the DNA methylation level was consistently diminished on one side, corresponding to the interior of the loop (FIGS. 7A-7B). This confirmed an observation first reported in (3). These results further confirmed the ability of Hi-Culfite to detect subtle effects that could be observed when methylation profiles at many loci are studied in aggregate.

TABLE 1
Quality metrics of Hi-Culfite libraries (two combined replicates) compared
to in situ Hi-C (eight combined replicates, Rao et al. Cell 2014)

| | Hi-Culfite GM12878 | Hi-C GM12878 |
|---|---|---|
| Sequenced Read Pairs: | 1,748,191,006 | 2,935,830,058 |

Alignment (% Sequenced Reads)

| | | | | |
|---|---|---|---|---|
| Normal Paired: | 1,092,961,004 | (62.52%) | 2,187,069,164 | (74.50%) |
| Chimeric Paired: | 518,160,223 | (29.64%) | 507,602,762 | (17.29%) |
| Chimeric Ambiguous: | 111,331,581 | (6.37%) | 110,359,127 | (3.76%) |
| Unmapped: | 25,738,198 | (1.47%) | 130,799,005 | (4.46%) |

Duplication and Complexity (% Sequenced Reads)

| | | |
|---|---|---|
| Alignable (Normal + Chimeric Paired): | 1,611,121,227 | 2,694,671,926 |
| Unique Reads: | 1,336,677,373 | 2,584,147,879 |
| PCR Duplicates: | 268,384,692 | 110,448,756 |
| Optical Duplicates: | 6,059,162 | 75,291 |

Analysis of Unique Reads (% Sequenced Reads/% Unique Reads)

| | | | | |
|---|---|---|---|---|
| Intra-fragment Reads: | 149,415,012 | (8.55%/12.18%) | 77,475,958 | (2.64%/3.00%) |
| Below MAPQ Threshold: | 186,326,300 | (10.66%/15.19%) | 238,800,841 | (8.13%/9.24%) |
| Hi-C Contacts: | 890,634,698 | (50.95%/72.62%) | 2,267,880,754 | (77.25%/87.76%) |

| | | |
|---|---|---|
| 3′ Bias (Long Range): | 56%-44% | 71%-29% |
| Pair Type % (L-I-O-R): | 25% - 25% - 25% - 25% | 25% - 25% - 25% - 25% |

Analysis of Hi-C Contacts (% Sequenced Reads/% Unique Reads)

| | | | | |
|---|---|---|---|---|
| Inter-chromosomal: | 247,760,751 | (14.17%/20.20%) | 577,717,760 | (19.68%/22.36%) |
| Intra-chromosomal: | 642,873,947 | (36.77%/52.42%) | 1,690,162,994 | (57.57%/65.41%) |
| Short Range (<20 Kb): | 200,014,362 | (11.44%/16.31%) | 539,732,380 | (18.38%/20.89%) |
| Long Range (>20 Kb): | 442,758,907 | (25.33%/36.10%) | 1,150,429,110 | (39.19%/44.52%) |

TABLE 2
Methylation analysis metrics for Hi-Culfite libraries prepared
from GM12878 and Hap1 cells treated for 8 days with DMSO
(control), 1 μM or 5 μM 5-azacytidine in DMSO.

| | | |
|---|---|---|
| Control | 1 μM | 5 μM |

GM12878 treated with 5-azacytidine

| | | | |
|---|---|---|---|
| C−>T conversion | 99.41% | 99.10% | 99.46% |
| CHH + CHG coverage | 73.84% | 73.11% | 72.91% |
| CpG C−>T conversion | 45.37% | 69.46% | 75.08% |
| CpG coverage | 71.59% | 70.71% | 70.30% |

Hap-1 treated with 5-azacytidine

| | | | |
|---|---|---|---|
| C−>T conversion | 99.06% | 99.37% | 98.82% |
| CHH + CHG coverage | 40.51% | 67.37% | 33.00% |
| CpG C−>T conversion | 37.66% | 86.16% | 85.79% |
| CpG coverage | 37.60% | 65.22% | 30.99% |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method for analyzing nucleic acids in cells, comprising:
fragmenting the nucleic acids, the fragmented nucleic acids comprising overhanging ends;
filling in the overhanging ends with one or more nucleotides comprising a label;
joining the filled-in ends to create one or more end-joined nucleic acid fragments with one or more junctions;
treating the end-joined nucleic acid fragments with bisulfite;
isolating the bisulfite-treated, end joined nucleic acid fragments using the label; and
determining sequence at the one or more junctions in the bisulfite treated, end-joined nucleic acid fragments, thereby determining spatial proximity between the nucleic acids and methylation profile of the nucleic acids.

2. The method of claim 1, further comprising determining a relationship between the spatial proximity and the methylation profile.

3. The method of claim 1, further comprising holding the nucleic acids in a fixed position relative to one another prior to fragmenting.

4. The method of claim 3, wherein the nucleic acids are held in the fixed position by crosslinking the cells or nuclei in the cells.

5. The method of claim 4, further comprising reversing the crosslinking.

6. The method of claim 1, further comprising isolating nuclei from the cells prior to fragmenting.

7. The method of claim 6, further comprising permeabilizing the nuclei.

8. The method of claim 1, wherein the nucleic acids are a part of chromatin.

9. The method of claim 1, wherein the nucleic acids are DNA.

10. The method of claim 1, wherein fragmenting is performed by digesting the nucleic acids using a nuclease.

11. The method of claim 10, wherein the nuclease is methylation insensitive.

12. The method of claim 1, further comprising, prior to the bisulfite treatment, shearing the nucleic acids.

13. The method of claim 12, wherein the sheared nucleic acids have a length from about 300 base pairs (bp) to about 500 bp.

14. The method of claim 1, wherein the bisulfite treated, end-joined nucleic acid fragments are isolated using a capture agent that binds to the nucleotides with the labeled.

15. The method of claim 14, wherein the capture agent is attached to a solid support.

16. The method of claim 15, wherein the solid support is a bead.

17. The method of claim 1, further comprising attaching one or more adaptors to the bisulfite treated, end-joined nucleic acid fragments.

18. The method of claim 17, wherein the one or more adaptors are attached after isolating the bisulfite treated, end-joined nucleic acid fragments.

19. The method of claim 1, further comprising amplifying the bisulfite treated, end joined nucleic acid fragments.

20. The method of claim 19, wherein the bisulfite treated, end-joined nucleic acid fragments are amplified using primers with one or more barcodes.

21. The method of claim 1, further comprising quantifying:
a frequency with which pairs of loci in the nucleic acids are found adjacent, and
a frequency with which loci in the nucleic acids are methylated.

22. The method of claim 1, wherein determining the spatial proximity between the nucleic acids comprises identifying chromosomal location of nucleic acid sequences both 5′ and 3′ of the junctions.

23. The method of claim 1, wherein determining the methylation profile comprises generating a genome-wide methylation profile of the cells.

24. The method of claim 1, further comprising correlating a relationship between the spatial proximity and the methylation profile with a disease.

25. The method of claim 1, wherein the sequence at one or more junctions in the bisulfite treated, end joined nucleic acid fragments is determined by transporting the fragments through an orifice in an electric field and measuring change of an electric current density across the orifice when the fragments are transported.

26. The method of claim 25, wherein the sequence at the one or more junctions in the bisulfite treated end-joined nucleic acid fragments is determined by nanopore sequencing.

27. A method of diagnosing a disease in a subject, comprising:
a. obtaining cells from the subject; and
b. analyzing nucleic acids in the cells according to the method of claim 1, wherein the spatial proximity and the methylation profile are indicative of the disease in the subject.

28. A method of treating a disease in a subject, comprising:
a. determining spatial proximity and methylation profile of a gene in a cell from the subject;
b. comparing the spatial proximity and the methylation profile to reference values, thereby identifying one or more nucleotides in the gene related to the disease; and
c. modifying at least one of the identified nucleotides.

29. The method of claim 28, wherein the spatial proximity and the methylation profile of the one or more identified nucleotides are indicative of the disease.

30. The method of claim 28, wherein modifying at least one of the identified nucleotides comprises modifying methylation of the at least one of the identified nucleotides.

31. The method of claim 28, wherein modifying at least one of the identified nucleotides comprises converting at least one of the identified nucleotides to another nucleotide.

32. A method for screening chemical libraries for agent modulating chromatin architecture and epigenetic profiles, comprising:
a. exposing cells to members of the chemical libraries;
b. determining the spatial proximity and methylation profile according to claim 1; and
c. comparing the spatial proximity and the methylation profile to spatial proximity and methylation profile of control cells, thereby identifying members in the chemical libraries that have effects on the spatial proximity and methylation profile.
